(19) United States
(12) Patent Application Publication
(10) Pub. No.: US 2022/0220485 A1
(43) Pub. Date: Jul. 14, 2022

(54) PD-L1 antisense oligonucleotides for use in tumor treatment

(71) Applicant: Secarna Pharmaceuticals GmbH & Co. KG, Marburg (DE)

(72) Inventors: Frank JASCHINSKI, Puchheim (DE); Richard KLAR, München (DE); Tamara THELEMANN, München (DE); Sven MICHEL, Bernried (DE)

(21) Appl. No.: 17/607,616

(22) PCT Filed: May 4, 2020
(86) PCT No.: PCT/EP2020/062264

§ 371 Date: Oct. 29, 2021

(30) Foreign Application Priority Data
May 3, 2019 (EP) 19172585.2

(51) Int. Cl. C12N 15/113; A61P 35/00

(52) U.S. Cl. CPC C12N 15/1138; A61P 35/00; C12N 2310/346; C12N 2310/3231; C12N 2310/315; C12N 2310/11

(57) ABSTRACT

The present invention refers to an oligonucleotide consisting of 10 to 20 nucleotides hybridizing with SEQ ID NO.1 encoding PD-L1, wherein the oligonucleotide has a fundamentally reduced number of potential off-target binding sites resulting in a markedly reduced risk for off-target effects. Further, the present invention is directed to a pharmaceutically composition comprising such oligonucleotide and a pharmaceutically acceptable excipient.

The present invention refers to an oligonucleotide consisting of 10 to 20 nucleotides hybridizing with SEQ ID NO.1 encoding PD-L1, wherein the oligonucleotide hybridizes with specific regions of SEQ ID NO.1 and the oligonucleotide has a fundamentally reduced number of potential off-target binding sites resulting in a markedly reduced risk for off-target effects. Further, the present invention is directed to a pharmaceutically composition comprising such oligonucleotide and a pharmaceutically acceptable excipient.

### TECHNICAL BACKGROUND

During the last decades of cancer research it became obvious that the immune system is indispensable to initiate and release an effective anti-tumor response. Therefore it needs to be integrated in common cancer therapies. However, cancer cells developed mechanisms to circumvent anti-tumor immune responses, e.g., by downregulating HLA molecules leading to impaired antigen presentation, by the secretion of inhibitory soluble mediators such as IL-10 or adenosine, or by expressing T cell inhibitory ligands.

The most prominent inhibitory ligands expressed on the surface of antigen presenting cells and cancer cells are Programmed cell death-ligand 1 and 2 (PD-L1/PD-L2). Programmed cell death-ligand 1 (PD-L1) also known as cluster of differentiation 274 (CD274) or B7 homolog 1 (B7-H1) is a protein that is encoded in humans by the CD274 gene. While PD-L2 (B7-DC or CD273) is expressed primarily on professional antigen presenting cells (such as B cells and dendritic cells), PD-L1 is expressed on non-lymphoid cells, such as parenchymal cells, virus-infected cells and tumor cells, as well as on other immune cells. The two ligands interact with their receptor Programmed cell death-1 (PD-1), expressed on several immune cells, such as activated T cells, B cells, natural killer cells and myeloid cells in the periphery.

In humans, genetic alterations of the PD-1 encoding gene (PDCD1) are associated with increased susceptibility towards several autoimmune diseases, such as systemic lupus erythematosus, type 1 diabetes, multiple sclerosis, rheumatoid arthritis, Grave's disease and ankylosing spondylitis. However, distinct from other negative immune regulators, PD-1 deficiency specifically and only affects antigen-specific autoimmune responses whereas deficiency of other negative regulators results in systemic, non-antigen-specific autoimmune phenotypes.

Until present, the blockade of PD-1/PD-L1 interactions by monoclonal antibodies or by genetic manipulation of PD-1 expression led to enhanced tumor eradication. Furthermore, clinical data suggest that enhanced PD-L1 expression in tumors correlates with poorer survival prognosis of different cancer patients. These results led to the development of several different fully humanized monoclonal antibodies targeting either PD-1 or PD-L1. Application of those antibodies showed positive response rates in humans in clinical trials of e.g., non-small-cell lung cancer, melanoma, renal cell carcinoma, and Hodgkin lymphoma with drug-related adverse events in a subset of patients. Nonetheless, therapeutic blockade of the PD-1 pathway is the most powerful target for immunological anti-tumor therapies in the clinics at present.

However, a large proportion of cancer patients (>70%) do not respond well to therapeutic blockade of PD-1 or PD-L1 using monoclonal antibody therapies. These data suggest the importance of accessing combinatorial therapies using agents to block additional negative or to activate positive regulators that might have additive and/or synergistic effects in order to improve antitumor immunotherapies. The application of antisense oligonucleotides targeting PD-L1 expression on mRNA level in combination with therapies that target other known negative (e.g., LAG-3; TIM-3; 2B4; CD160) or positive (e.g., CD137; CD40) immune-regulatory pathways could provide better therapeutic efficacy than targeting the PD-1/PD-L1 pathway alone. Several studies indicate the presence of an immune inhibitory soluble form of PD-L1 (sPD-L1) in sera of cancer patients, correlating with disease severity and a negative patient survival outcome. Thus, it is very likely that the soluble form of PD-L1 cannot be fully captured by conventional monoclonal antibodies directed against PD-L1 on a systemic level.

Furthermore, antibodies are huge in molecular size and therefore might not reach targets expressed on dense and packed tissues as it is the case for many different tumors. Thus, while targeting PD-L1 appears to be a promising approach to develop and improve novel immunotherapies against different cancers, no satisfactory solution for achieving that has yet been found. Hence, there is still a high scientific and medical need for therapeutic agents, which reduce or inhibit PD-L1 expression and/or activity. Thus, the inhibition of target expression could be a more promising approach to develop and improve novel immunotherapies against different cancers than conventional antibody therapies. Currently two competing technologies are predominantly used for specific suppression of mRNA expression: Antisense oligonucleotides and siRNA.

Due to its double stranded nature, siRNA does not cross the cell membrane by itself and delivery systems are required for its activity in vitro and in vivo. While delivery systems for siRNA exist that efficiently deliver siRNA to liver cells in vivo, there is currently no system that can deliver siRNA in vivo to extra-hepatic tissues such as tumors with sufficient efficacy. Therefore, siRNA approaches to target PD-L1 are currently limited to ex vivo approaches, for example for the generation of dendritic cell-based tumor vaccines. For antisense oligonucleotides efficacy in cell culture is typically determined after transfection using transfection reagents or electroporation. Antisense approaches directed against PD-L1 are described, for example, in WO 2006/042237 or WO 2016/057933, or in Mazanet et al., J. Immunol. 169 (2002) 3581-3588. Moreover, it was recently discovered that antisense oligonucleotides that are modified by so called 3^{rd} generation chemistries, such as 2′,4′-LNA (see, for example, WO 2014/154843 A1) or constrained ethyl bridged nucleic acids (c-ET), can enter cells in vitro and in vivo without a delivery system to achieve target downregulation. Additionally, double-stranded RNA molecules (see WO 2011/127180) and so-called “3rd generation antisense compounds”, which comprise two antisense constructs linked via their 5′ ends (see WO 2016/138278), have been tested as PD-L1 inhibitors.

However, in some approaches described in the prior art only moderate target suppression levels were achieved and relatively high concentrations of oligonucleotides were required for efficient target suppression. For example, in U.S. Pat. No. 8,563,528 a concentration of 10 μM resulted in a target inhibition of just 70%. IC₅₀ values for 3^{rd} generation oligonucleotides without transfection reagent typically range between 300 and 600 nM (Zhang et al. Gene Therapy (2011) 18, 326-333). After systemic administration in vivo, only relatively low oligonucleotide concentrations can be achieved in relevant target tissues. Therefore antisense oligonucleotides that reach high maximal target suppression at low concentration would clearly result in an enhanced therapeutic effect. WO 2018/065589 A1 and WO 2017/157899 A1 describe 3rd generation antisense oligonucleotides showing inhibition of PD-L1 expression as approach to develop and improve novel immunotherapies against different cancers.

However, there is still a need for improved oligonucleotides, in particular antisense oligonucleotides having increased specificity in the binding to the target region and thus, significantly reduced side effects resulting amongst others in reduced toxicity for use in efficient prevention and/or treatment of tumor diseases.

### SUMMARY

The present invention refers to an oligonucleotide comprising or consisting of 10 to 20 nucleotides hybridizing with SEQ ID NO.1 (GRCh38_9_5447492_5473576) encoding PD-L1, wherein the oligonucleotide hybridizes within the region of from position 15400 to position 22850 of SEQ ID NO.1 or within the region of from position 3100 to position 19500 of SEQ ID NO.1. Without further selection there is a potential that the oligonucleotide does not only bind to the intended target sequence but additionally to other sequences showing a certain sequence complementarity resulting in an increased risk for off-target effects. The oligonucleotides of the present invention are selected to strongly reduce this risk.

Optionally, the oligonucleotide of the present invention does only bind to the target RNA with zero mismatches. There is no off-target RNA where the oligonucleotide can bind with zero or one mismatch and there are at max. 20 off-targets where the oligonucleotide can bind with two mismatches.

The oligonucleotide of the present invention comprises for example one or more modified nucleotides. The oligonucleotide comprises for example a LNA, a c-ET, an ENA, a polyalkylene oxide-, a 2′-fluoro-, a 2′-O-methoxy-, a FANA and/or a 2′-O-methyl-modified nucleotide. The modified nucleotide(s) is/are located for example at the 5′- or 3′-end, or at the 5′- and 3′-end of the oligonucleotide.

The oligonucleotide of the present invention comprises for example a sequence selected from the group consisting of SEQ ID NO.2, SEQ ID NO.3, SEQ ID NO.4, SEQ ID NO.5, SEQ ID NO.6, SEQ ID NO.7, SEQ ID NO.8, SEQ ID NO.9, SEQ ID NO.10, SEQ ID NO.11, SEQ ID NO.12 and a combination thereof, or the oligonucleotide of the present invention comprises for example a sequence selected from the group consisting of SEQ ID NO.13, SEQ ID NO.14, SEQ ID NO.15, SEQ ID NO.16, SEQ ID NO.17, SEQ ID NO.18, SEQ ID NO.19, SEQ ID NO.20, SEQ ID NO.21, SEQ ID NO.22, SEQ ID NO.23, SEQ ID NO.24, SEQ ID NO.25, SEQ ID NO.26, SEQ ID NO.27, SEQ ID NO.28, SEQ ID NO.29, SEQ ID NO.30, SEQ ID NO.31, SEQ ID NO.32, SEQ ID NO.33, SEQ ID NO.34, SEQ ID NO.35, SEQ ID NO.36, SEQ ID NO.37, SEQ ID NO.38, SEQ ID NO.39, SEQ ID NO.40, SEQ ID NO.41, SEQ ID NO.42, SEQ ID NO.43, SEQ ID NO.44, SEQ ID NO.45, SEQ ID NO.46, SEQ ID NO.47, SEQ ID NO.48, SEQ ID NO.49, SEQ ID NO.50, SEQ ID NO.51, SEQ ID NO.52, SEQ ID NO.53, SEQ ID NO.54, SEQ ID NO.55, SEQ ID NO.56, SEQ ID NO.57, SEQ ID NO.58, SEQ ID NO.59, SEQ ID NO.60, SEQ ID NO.61, SEQ ID NO.62, SEQ ID NO.63, SEQ ID NO.64, SEQ ID NO.65, SEQ ID NO.66, SEQ ID NO.67, SEQ ID NO.68, SEQ ID NO.69, SEQ ID NO.70, SEQ ID NO.71, SEQ ID NO.72, SEQ ID NO.73, SEQ ID NO.74, SEQ ID NO.75, SEQ ID NO.76 and a combination thereof.

Alternatively, the oligonucleotide of the present invention comprises a sequence selected from the group consisting of SEQ ID NO.2, SEQ ID NO.3, SEQ ID NO.4, SEQ ID NO.5, SEQ ID NO.6, SEQ ID NO.7, SEQ ID NO.8, SEQ ID NO.9, SEQ ID NO.10, SEQ ID NO.11, SEQ ID NO.12, SEQ ID NO.13, SEQ ID NO.14, SEQ ID NO.15, SEQ ID NO.16, SEQ ID NO.17, SEQ ID NO.18, SEQ ID NO.19, SEQ ID NO.20, SEQ ID NO.21, SEQ ID NO.22, SEQ ID NO.23, SEQ ID NO.24, SEQ ID NO.25, SEQ ID NO.26, SEQ ID NO.27, SEQ ID NO.28, SEQ ID NO.29, SEQ ID NO.30, SEQ ID NO.31, SEQ ID NO.32, SEQ ID NO.33, SEQ ID NO.34, SEQ ID NO.35, SEQ ID NO.36, SEQ ID NO.37, SEQ ID NO.38, SEQ ID NO.39, SEQ ID NO.40, SEQ ID NO.41, SEQ ID NO.42, SEQ ID NO.43, SEQ ID NO.44, SEQ ID NO.45, SEQ ID NO.46, SEQ ID NO.47, SEQ ID NO.48, SEQ ID NO.49, SEQ ID NO.50, SEQ ID NO.51, SEQ ID NO.52, SEQ ID NO.53, SEQ ID NO.54, SEQ ID NO.55, SEQ ID NO.56, SEQ ID NO.57, SEQ ID NO.58, SEQ ID NO.59, SEQ ID NO.60, SEQ ID NO.61, SEQ ID NO.62, SEQ ID NO.63, SEQ ID NO.64, SEQ ID NO.65, SEQ ID NO.66, SEQ ID NO.67, SEQ ID NO.68, SEQ ID NO.69, SEQ ID NO.70, SEQ ID NO.71, SEQ ID NO.72, SEQ ID NO.73, SEQ ID NO.74, SEQ ID NO.75, SEQ ID NO.76 and a combination thereof.

The oligonucleotide of the present invention is for example selected from the group consisting of oligonucleotides of Table 1, of Table 2 and a combination thereof. The present invention further relates to a pharmaceutical composition comprising an oligonucleotide of the present invention and a pharmaceutically acceptable excipient. The oligonucleotide of the present invention, the pharmaceutical composition of the present invention, or a combination thereof is for example used in a method of preventing and/or treating a disease or disorder selected from the list of a malignant tumor, and a benign tumor. The tumor is for example selected from the group consisting of solid tumors, blood born tumors, leukemia, tumor metastasis, hemangiomas, acoustic neuromas, neurofibroma, trachoma, pyogenic granulomas, psoriasis, astrocytoma, blastoma, Ewing's tumor, craniopharyngioma, ependymoma, medulloblastoma, glioma, hemangioblastoma, Hodgkin's lymphoma, mesothelioma, neuroblastoma, non-Hodgkin's lymphoma, pinealoma, retinoblastoma, sarcoma, seminoma, and Wilms' tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF THE FIGURES

FIG. 1A and FIG. 1B depict an efficiency screening of oligonucleotides of the present invention in HDLM-2 and MDA-MB-231 cells testing the inhibition of PD-L1 expression. PD-L1 expression values were normalized to HPRT1 expression values and set in relation to mock-treated cells. FIG. 1A shows inhibition of PD-L1 expression in HDLM-2 cells and FIG. 1B shows inhibition of PD-L1 expression in MDA-MB-231 cells after administration of antisense oligonucleotides of the present invention.

FIG. 2 depicts dose-dependent inhibition of PD-L1 mRNA expression after administration of antisense oligonucleotides A03062H (SEQ ID NO.6), A0306311 (SEQ ID NO.7), A03077HI, A03084HI (SEQ ID NO.27), A03107HI (SEQ ID NO.49) and A03108HI (SEQ ID NO.50), respectively, of the present invention in HDML-2 cells. Each oligonucleotide was administered in concentrations of 10 μM, 2.5 μM, 625 nM, 157 nM, 39 nM, 10 nM, 2.5 nM.

FIG. 3 shows liver toxicity testing of A03063H (SEQ ID NO.7) and A03108HI (SEQ ID NO.50) of the present invention in comparison to antisense oligonucleotides of prior art hybridizing with PD-L1 mRNA. Toxicity was tested after 5, 9 and 12 days, wherein toxicity of the oligonucleotides of the present invention is very low.

FIG. 4 depicts a schematic presentation of the mismatch test showing the number of an oligonucleotide with a length of n nucleotides that binds to an off-target with zero mismatches (0 mm, meaning that the oligonucleotide has 100% sequence homology to the off-target nucleotide sequence), one mismatch (1 mm, meaning that the oligonucleotide has ((n−1)/n*100) % sequence homology to the off-target nucleotide sequence) or with two mismatches (2 mm, meaning that the oligonucleotide has ((n−2)/n*100) % sequence homology to the off-target nucleotide sequence).

FIG. 5A and FIG. 5B shows dose-dependent PD-L1 protein knockdown of ASO A03063H (SEQ ID NO.7) and A03108HI (SEQ ID NO.50) in HDLM-2 cells (FIG. 5A) and MiaPaCa cells (FIG. 5B).

FIG. 6 depicts PD-L1 protein knockdown in dendritic cells using ASO A03063H (SEQ ID NO.7) and A03108HI (SEQ ID NO.50), respectively.

FIGS. 7A and 7B show persistency of PD-L1 protein knockdown in HDLM-2 cells using ASO A03063H (SEQ ID NO.7) and A03108HI (SEQ ID NO.50), respectively. FIG. 7A proofs the rapid proliferation of HDLM-2 cells and FIG. 7B shows the effect of ASO A03063H (SEQ ID NO.7) or A03108HI (SEQ ID NO.50) on PD-L1 expression.

### DETAILED DESCRIPTION

The present invention provides a successful inhibitor of PD-L1 expression, wherein the inhibitor is a human oligonucleotide such as an antisense oligonucleotide hybridizing with the pre-mRNA sequence of PD-L1 of SEQ ID NO.1 (GRCh38_9_5447492_5473576) and inhibiting the expression and activity, respectively, of PD-L1. pre-mRNA of SEQ ID NO.1 comprises exons and introns of PD-L1. The oligonucleotides of the present invention hybridize either with an exon region or an intron region of SEQ ID NO.1 and hybridizes very target specific. Thus, the oligonucleotides of the present invention represent an interesting and highly efficient tool for use in a method of preventing and/or treating disorders, where the PD-L1 expression and activity, respectively, is increased as the oligonucleotides have a very low off-target effect and consequently, significantly reduced side effect and significantly reduced toxicity.

An oligonucleotide of the present invention hybridizes within the region of from position 15400 to position 22850 of SEQ ID NO.1 or within the region of from position 3100 to position 19500 of SEQ ID NO.1, wherein the starting and end point of the region, i.e., position 3100, 15400, 19500 and 22850 are comprised by the region.

The oligonucleotide of the present invention has inhibitor function, i.e., it inhibits the transcription and expression, respectively, of PD-L1. Inhibition according to the present invention comprises any level of reduction of the transcription and expression, respectively, of PD-L1 for example in comparison to a cell without administration of an oligonucleotide such as an antisense nucleotide hybridizing with PD-L1.

An oligonucleotide with a length of n nucleotides of the present invention does not bind to any off-target nucleotide sequence with 100% sequence complementarity (i.e., the oligonucleotide has zero mismatches to any off-target nucleotide sequence), nor does it bind to any off-target nucleotide sequence with ((n−1)/n*100) % sequence complementarity (i.e., the oligonucleotide has one mismatch to any off-target sequence). An oligonucleotide of the present invention binds only to a very limited number of off-target nucleotide sequences with ((n−2)/n*100) % sequence complementarity (i.e., the oligonucleotide has two mismatches to the respective off-target nucleotide sequence). Oligonucleotides of the present invention fulfilling these conditions have therefore a significantly reduced risk to induce off-target effects in comparison to oligonucleotides hybridizing with PD-L1 pre-mRNA of SEQ ID NO.1, but not fulfilling these conditions. Oligonucleotides of the present invention hybridize for example with the region of from position 15400 to position 22850 of SEQ ID NO.1 or with the region of from position 3100 to position 19500 of SEQ ID NO.1. An oligonucleotide hybridizing with one of these regions, but not fulfilling the above mentioned strict conditions regarding the mismatches according to the present invention does not present the significantly reduced risk to induce off-target effects as oligonucleotides of the present invention. The number of off-target sites of an oligonucleotide of the present invention binding to off-target nucleotide sequence with ((n−2)/n*100) % sequence complementarity is limited to max. 5, max. 10, max. 15, max. 20, max. 25, max. 30, max. 35 or max. 40 off-target bindings and effects, respectively.

An off-target effect is a biological activity of an oligonucleotide that is different from and not at that of its intended position and/or biological target. An off-target effect comprises for example the binding of an oligonucleotide such as an antisense oligonucleotide (ASO) to a different position at the nucleic acid sequence or to a different target nucleic acid sequence. The off-target effect is intended or unintended and has for example a physiological and/or biochemical effect or is silent, i.e., does not have any or at least not a measurable effect on the cell, tissue, organ and/or organism. It contributes for example to side effects such as toxicity.

Antisense oligonucleotides have significant advantages in comparison to siRNA. Antisense oligonucleotides can be transfected without transfecting reagent in vitro and thus, the transfection is closer to in vivo conditions than transfections using transfecting reagents which are obligatory for the transfection of siRNA. In vivo systemic administration of antisense oligonucleotides is possible in different tissues whereas the administration of siRNA in vivo is dependent on delivery systems such as GalNAc for example in liver. Moreover, antisense oligonucleotides are shorter than siRNA and therefore, are less complex in synthesis and in the uptake into cells. siRNA regularly show off-target effects of passenger strands which likewise can initiate siRNA. Passenger strand RISC loading is a significant concern for RNAi drugs because the passenger strand could direct RNAi activity towards unintended targets, resulting in toxic side effects (see Chackalamannil, Rotella, Ward, Comprehensive Medicinal Chemistry III Elsevier, Mar. 6, 2017). Antisense oligonucleotides do not comprise a passenger strand.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides (ASO) consisting of or comprising 10 to 25 nucleotides, 12 to 22 nucleotides, 15 to 20 nucleotides, 16 to 18 nucleotides, or 15 to 17 nucleotides. The oligonucleotides for example consist of or comprise 10, 11, 12, 13, 14, 15, 16, 17, 18, 19, 20 or 25 nucleotides.

The oligonucleotide of the present invention comprises at least one nucleotide which is modified. The modified nucleotide is for example a bridged nucleotide such as a locked nucleic acid (LNA, e.g., 2′,4′-LNA), cET, ENA, a polyalkylene oxide-, a 2′-fluoro-, a 2′-O-methoxy-, a FANA and/or a 2′-O-methyl-modified nucleotide or a combination thereof. In some embodiments, the oligonucleotide of the present invention comprises nucleotides having the same or different modifications. In some embodiments the oligonucleotide of the present invention comprises a modified phosphate backbone, wherein the phosphate is for example a phosphorothioate.

The oligonucleotide of the present invention comprises the one or more modified nucleotide for example at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more unmodified nucleotides. The following Tables 1 and 2 present embodiments of oligonucleotides comprising modified nucleotides for example LNA which are indicated by (+) and phosphorothioate (PTO) indicated by (*) and bind with the first nucleotide to a given position in GRCh38_9:5447492-5473576 indicated by (s). The oligonucleotides consisting of or comprising the sequences of Tables 1 and 2, respectively, may comprise any other modified nucleotide and any other combination of modified and unmodified nucleotides. Oligonucleotides of Table 1 hybridize with exonic regions of the pre-mRNA of human PD-L1:

TABLE 1
List of antisense oligonucleotides hybridizing with exonic
regions of human PD-L1 pre-mRNA for example of SEQ ID NO.
1; Neg1 is an oligonucleotide representing a negative
control which is not hybridizing with PD-L1 of SEQ ID NO.
1. “H” means “human exonic region” and indicates an oligo-
nucleotide primarily hybridizing with exonic regions of
the pre-mRNA of human PD-L1.

| | | | | SEQ |
|---|---|---|---|---|
| | | Antisense | Antisense Sequence 5′-3′ | ID |
| Position^{§} | Name | Sequence 5′-3′ | with PTO (*) and LNA (+) | NO. |
| 15465 | A03058H | TTACCACTCAGGACTTG | +T*+T+A*C*C*A*C*T*C*A*G* | 2 |
| | | | G*A*C*+T*+T*+G | |
| 20363 | A03059H | ATGCTGGATTACGTCTC | +A*+T+G*C*T*G*G*A*T*T*A* | 3 |
| | | | C*G*T*+C*+T+C | |
| 20364 | A03060H | AATGCTGGATTACGTCT | +A*+A*+T*G*C*T*G*G*A*T*T* | 4 |
| | | | A*C*G*+T+C*+T | |
| 20655 | A03061H | ATGATTTGCTTGGAGGC | +A*+T*G*+A*T*T*T*G*C*T*T* | 5 |
| | | | G*G*+A*G*G*+C | |
| 20911 | A03062H | GGCGACAAAATTGTAAC | +G*+G*+C*G*A*C*A*A*A*A*T* | 6 |
| | | | T*G*T*+A*+A*+C | |
| 20920 | A03063H | GTTTAGTTTGGCGACAA | +G*+T*+T*T*A*G*T*T*T*G*G* | 7 |
| | | | C*G*A*C*+A*+A | |
| 22496 | A03064H | CACAACGAATGAGGCTT | +C*+A*+C*A*A*C*G*A*A*T*G* | 8 |
| | | | A*G*G*+C*+T+T | |
| 22799 | A03065H | TAGACTATGTGCCTTGC | +T+A*+G*A*C*T*A*T*G*T*G* | 9 |
| | | | C*C*T*+T*+G*+C | |
| 22803 | A03066H | TGAGTAGACTATGTGCC | +T+G*+A*G*T*A*G*A*C*T*A* | 10 |
| | | | T*G*T*+G*+C*+C | |
| 20360 | A03067H | TGGATTACGTCTCCTC | +T+G*+G*A*T*T*A*C*G*T*C* | 11 |
| | | | T*C*+C*+-T+C | |
| 20363 | A03068H | TGCTGGATTACGTCTC | +T+G*+C*T*G*G*A*T*T*A*C* | 12 |
| | | | G*T+C*+T+C | |
| | Neg1 | | +C*+G*+T*T*T*A*G*G*C*T* | 77 |
| | | | A*T*G*T*A*+C*+T+T | |

Oligonucleotides of Table 2 hybridize with intronic regions of the pre-m RNA of human PD-L1:

TABLE 2
List of antisense oligonucleotides hybridizing with intronic regions of the
human PD-L1 pre-mRNA; Neg1 (SEQ ID NO. 77) and R01011 (SEQ ID NO. 78),
respectively, is an oligonucleotide representing a negative control which is
not hybridizing with PD-L1 of SEQ ID NO. 1. “HI” indicates that the oligo-
nucleotides hybridizes with an intron.

| | | | | SEQ |
|---|---|---|---|---|
| | | Antisense | Antisense Sequence 5′-3′ with | ID |
| Position§ | Name | Sequence 5′-3′ | PTO (*) and LNA (+) | NO. |
| 3145 | A03069HI | CGAGCTAGCCAGAGATA | +C*+G*+A*G*C*T*A*G*C*C*A*G*A*G*+A*+T+A | 13 |
| 3229 | A03070HI | CTAGACCATCGCGTT | +C*+T+A*G*A*C*C*A*T*C*G*C*+G*+T*+T | 14 |
| 3301 | A03071HI | AATCGCGCCTGGAGGAA | +A*+A*+T*C*G*C*G*C*C*T*G*G*A*G*+G*+A*+A | 15 |
| 3306 | A03072HI | ACTGAATCGCGCCTGG | -w+C*+T*G*A*A*T*C*G*C*G*C*C*+T*+G*+G | 16 |
| 3366 | A03073HI | TACCTATCCTATACTAC | +T+A*+C*C*T*A*T*C*C*T*A*TkA*C*+T*+A*+C | 17 |
| 3530 | A03074HI | TGGACCTGCTTAGCGCA | +T+G*+G*A*C*C*T*G*C*T*T*A*G*C*+G*+C*+A | 18 |
| 4509 | A03075HI | ACCGGTTAAACTTCCTT | +A*+C*C*G*G*T*T*A*A*A*C*T*T*C*C*+T+T | 19 |
| 5303 | A03076HI | TATGGCCTACTCTGGTG | +T+A*+T*G*G*C*C*T*A*C*T*C*T*G*+G*+-T+G | 20 |
| 5639 | A03077HI | GGAATAGCGATGGCATT | +G*+G*+A*A*T*A*G*C*G*A*T*G*G*C*+A*+T+T | 21 |
| 5659 | A03078HI | TCCGTCTATCCTGTAGA | +T+C*+C*G*T*C*T*A*T*C*C*T*G*T*+A*+G*+A | 22 |
| 5659 | A03079HI | TCCGTCTATCCTGTAGA | +T+C*C*G*T*C*T*A*T*C*C*T*G*T*A*+G*+A | 22 |
| 6580 | A03080HI | TCCAAACTGACGTAGAA | +T*+C*+C*A*A*A*C*T*G*A*C*G*T*A*+G*+A*+A | 23 |
| 6654 | A03081HI | CACCTTACCAAACCGTA | +C*+A*+C*C*T*T*A*C*C*A*A*A*C*C*+G*+T+A | 24 |
| 7214 | A03082HI | ATCGTAAATGCGGATGT | +A*-FT+C*G*T*A*A*A*T*G*C*G*G*A*+T*+G*+T | 25 |
| 7224 | A03083HI | TCCTATTACAATCGTAA | +T+C*+C*T*A*T*T*A*C*A*A*T*C*G*+T*+A*+A | 26 |
| 7340 | A03084HI | GAGCTTGACCACAATTG | +G*+A*+G*C*T*T*G*A*C*C*A*C*A*A*+T+T*+G | 27 |
| 7568 | A03085HI | ATCGATGCCACGTATAT | +A*+T*+C*G*A*T*G*C*C*A*C*G*T*A*+T+A*+T | 28 |
| 7573 | A03086HI | ACTAATCGATGCCACG | +A*+C*+T*A*A*T*C*G*A*T*G*C*C*+A*+C*+G | 29 |
| 7575 | A03087HI | TCAACTAATCGATGCCA | +T*+C*+A*A*C*T*A*A*T*C*G*A*T*G*+C*+C*+A | 30 |
| 8355 | A03088HI | TAGTTACAGGCCGTGAA | +T+A*+G*T*T*A*C*A*G*G*C*C*G*T*+G*+A*+A | 31 |
| 8357 | A03089HI | TATAGTTACAGGCCGTG | +T+A*+T*A*G*T*T*A*C*A*G*G*C*C*+G*+T*+G | 32 |
| 8826 | A03090HI | TCATTGCGTAAAGTAGA | +T*+C*+A*T*T*G*C*G*T*A*A*A*G*T*+A*+G*+A | 33 |
| 9487 | A03091HI | TATCTGGTCGGTTATGT | +T*+A*+T*C*T*G*G*T*C*G*G*T*T*A*+T*+G*+T | 34 |
| 9494 | A03092HI | ATCACTTTATCTGGTCG | +A*+T+C*A*C*T*T*T*A*T*C*T*G*G*+T+C*+G | 35 |
| 9508 | A03093HI | CACAGCGTTTATAAATC | +C*+A*+C*A*G*C*G*T*T*T*A*T*A*A*+A*+T*+C | 36 |
| 9513 | A03094HI | ATTGGCACAGCGTTTAT | +A*+T+T*G*G*C*A*C*A*G*C*G*TkT+T+A*+T | 37 |
| 10375 | A03095HI | ACTGACGGACCTAATAA | +A*+C*+T*G*A*C*G*G*A*C*C*T*A*A*+T+A*+A | 38 |
| 10623 | A03096HI | CCGAGGAACTAACACTC | +C*+C*+G*A*G*G*A*A*C*T*A*A*C*A*+C*+T+C | 39 |
| 10625 | A03097HI | TACCGAGGAACTAACAC | +T*+A*+C*C*G*A*G*G*A*A*C*T*A*A*+C*+A*+C | 40 |
| 10631 | A03098HI | GGTCAATACCGAGGAAC | +G*+G*+T*C*A*A*T*A*C*C*G*A*G*G*+A*+A*+C | 41 |
| 10833 | A03099HI | ACGCCATTGCAGGAAAT | +A*+C*+G*C*C*A*T*T*G*C*A*G*G*A*+A*+A*+T | 42 |
| 11038 | A03100HI | GATTGATGGTAGTTAGC | +G*+A*+T*T*G*A*T*G*G*T*A*G*T*T*+A*+G*+C | 43 |
| 11353 | A03101HI | GCCTGATATTTGCGGAT | +G*+C*+C*T*G*A*T*A*T*T*T*G*C*G*G*+A*+T | 44 |
| 11656 | A03102HI | ATCAGTGCCGGAAGATT | +A*+T*+C*A*G*T*G*C*C*G*G*A*A*G*+A*+T*+T | 45 |
| 11656 | A03103HI | ATCAGTGCCGGAAGATT | +A*+T*C*A*G*T*G*C*C*G*G*A*A*G*+A*+T*+T | 45 |
| 11659 | A03104HI | ACCATCAGTGCCGGAAG | +A*+C*+C*A*T*C*A*G*T*G*C*C*G*G*+A*+A*+G | 46 |
| 11865 | A03105HI | GGTTGCCTTGTTCTAAG | +G*+G*+T*T*G*C*C*T*T*G*T*T*C*T*+A*+A*+G | 47 |
| 11975 | A03106HI | TTGCATATGGAGGTGAC | +T+T+G*C*A*T*A*T*G*G*A*G*G*T*+G*+A*+C | 48 |
| 12040 | A03107HI | AAGCTATGTTACCACAC | +A*+A*+G*C*T*A*T*G*T*T*A*C*C*A*+C*+A*+C | 49 |
| 12243 | A03108HI | GTTAGGTTCAGCGATTA | +G*+T*+T*A*G*G*T*T*C*A*G*C*G*A*+T*+T*+A | 50 |
| 12244 | A03109HI | TGTTAGGTTCAGCGATT | +T+G*+T*T*A*G*G*T*T*C*A*G*C*G*+A*+-1*+T | 51 |
| 12372 | A03110HI | ACAGCAGTCGATTTGGT | +A*+C*+A*G*C*A*G*T*C*G*A*T*T*T*+G*+G*+T | 52 |
| 12377 | A03111HI | GAATGACAGCAGTCGAT | +G*+A*+A*T*G*A*C*A*G*C*A*G*T*C*+G*+A*+T | 53 |
| 12816 | A03112HI | ACTCGATAGTAGCAGTA | +A*+C*+T*C*G*A*T*A*G*T*A*G*C*A*+G*+T*+A | 54 |
| 12820 | A03113HI | AGTACTCGATAGTAGC | +A*+G*+T*A*C*T*C*G*A*T*A*G*T*+A*+G*+C | 55 |
| 12823 | A03114HI | TAGTAGTACTCGATAGT | +T*+A*+G*T*A*G*T*A*C*T*C*G*A*T*+A*+G*+T | 56 |
| 12826 | A03115HI | TTGTAGTAGTACTCGAT | +T+T+G*T*A*G*T*A*G*T*A*C*T*C*+G*+A*+T | 57 |
| 12834 | A03116HI | AGTGCTAATTGTAGTAG | +A*+G*+T*G*C*T*A*A*T*T*G*T*A*G*+T+A*+G | 58 |
| 13193 | A03117HI | ATACGTACACCAGAGGT | +A*+T+A*C*G*T*A*C*A*C*C*A*G*A*+G*+G*+T | 59 |
| 13667 | A03118HI | AGACCTCGCAGTGTTAT | +A*+G*+A*C*C*T*C*G*C*A*G*T*G*T*+T+A*+T | 60 |
| 13670 | A03119HI | ATTAGACCTCGCAGTGT | +A*+T+T*A*G*A*C*C*T*C*G*C*A*G*+T+G*+T | 61 |
| 13676 | A03120HI | TACTTAATTAGACCTCG | +T*+A*+C*T*T*A*A*T*T*A*G*A*C*C*+T*+C*+G | 62 |
| 13709 | A03121HI | TATCGGCCACTGTATGA | +T*+A*+T*C*G*G*C*C*A*C*T*G*T*A*T*+G*+A | 63 |
| 14271 | A03122HI | GATTAAGATACGTAGT | +G*+A*+T*T*A*A*G*A*T*A*C*G*T*+A*+G*+T | 64 |
| 14504 | A03123HI | CATAACTAAGGACGTT | +C*+A*+T*A*A*C*T*A*A*G*G*A*C*+G*+T+T | 65 |
| 14508 | A03124HI | ATCGTCATAACTAAGGA | +A*+T*+C*G*T*C*A*T*A*A*C*T*A*A*+G*+G*+A | 66 |
| 14658 | A03125HI | TATGTTCCTGGTGATAC | +T+A*+T*G*T*T*C*C*T*G*G*T*G*A*+T*+A*+C | 67 |
| 15948 | A03126HI | CATGGTGTTGGATTGCC | +C*+A*+T*G*G*T*G*T*T*G*G*A*T*T*+G*+C*+C | 68 |
| 16300 | A03127HI | CTGTTGCTAATCTGACC | +C*+T+G*T*T*G*C*T*A*A*T*C*T*G*+A*+C*+C | 69 |
| 16598 | A03128HI | ACACCGTCCTGGATTAT | +A*+C*+A*C*C*G*T*C*C*T*G*G*A*T*+-1*+A*+T | 70 |
| 16608 | A03129HI | TCTGTTCACAACACCGT | +T+C*+T*G*T*T*C*A*C*A*A*C*A*C*+C*+G*+T | 71 |
| 16674 | A03130HI | AATACCTGAGGACTCGT | +A*+A*+T*A*C*C*T*G*A*G*G*A*C*T*+C*+G*+T | 72 |
| 17272 | A03131HI | CTAGTAGCCTACAGTAC | +C*+T+A*G*T*A*G*C*C*T*A*C*A*G*+T*+A*+C | 73 |
| 17667 | A03132HI | GCTTGCACAGTACCACA | +G*+C*+T*T*G*C*A*C*A*G*T*A*C*C*+A*+C*+A | 74 |
| 17859 | A03133HI | CTGGAATGGCGAGATAC | +C*+T+G*G*A*A*T*G*G*C*G*A*G*A*+T+A*+C | 75 |
| 19480 | A03134HI | TCAGACGGTGGAGGAGT | +T*+C*+A*G*A*C*G*G*T*G*G*A*G*G*+A*+G*+T | 76 |
| 19480 | A03135HI | TCAGACGGTGGAGGAGT | +T+C*+A*G*A*C*G*G*T*G*G*A*G*G*A*+G*+T | 76 |
| | Neg1 | | +C*+G*+T*T*T*A*G*G*C*T*A*T*G*T*A*+C*+T*+T | 77 |
| | R01011 | | +G*+A*+T*C*A*T*T*C*G*C*G*G*A*C*A*C*+A*+A*+C | 78 |

Table 1 and Table 2 present antisense sequences 5′-3′ of the oligonucleotides of the present invention, which comprise different modifications of nucleotides such as LNA-modification. In these Tables LNA-modified nucleotides are indicated by “+” and a phosphorothioate is indicated by “*”.

The oligonucleotide of the present invention inhibits for example at least about 50%, 55%, 60%, 65%, 70%, 75%, 80%, 85%, 90%, 92%, 94%, 95%, 96%, 97%, 98%, 99% or 100% of the expression of PD-L1 such as the, e.g., human, rat or murine, PD-L1 expression.

The oligonucleotide of the present invention inhibits the expression of PD-L1 at a nanomolar or micromolar concentration for example in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is for example used in a concentration of 1, 3, 5, 9, 10, 15, 27, 30, 40, 50, 75, 82, 100, 250, 300, 500, or 740 nM, or 1, 2.2, 3, 5, 6.6 or 10 μM.

The PD-L1 oligonucleotide of the present invention is for example administered once or repeatedly, e.g., every 12 h, every 24 h, every 48 h for some weeks, months or years, or it is administered every week, every two weeks, every three weeks or every months.

The present invention further refers to a pharmaceutical composition comprising an oligonucleotide of the present invention and for example a pharmaceutically acceptable carrier, excipient and/or dilutant. In some embodiments, the pharmaceutical composition further comprises a chemotherapeutic, another oligonucleotide, an antibody, a small molecule or a combination thereof.

The oligonucleotide or the pharmaceutical composition of the present invention is for example for use in a method of preventing and/or treating a disorder. In some embodiments, the use of the oligonucleotide or the pharmaceutical composition of the present invention in a method of preventing and/or treating a disorder is combined with radiotherapy. The radiotherapy may be further combined with a chemotherapy (e.g., platinum, gemcitabine). The disorder is for example characterized by a PD-L1 imbalance, i.e., the PD-L1 level is increased in comparison to the level in a normal, healthy cell, tissue, organ or subject. The PD-L1 level is for example increased by an increased PD-L1 expression and activity, respectively. The PD-L1 level can be measured by any standard method such as immunohistochemistry, western blot, quantitative real time PCR or QuantiGene assay known to a person skilled in the art.

An oligonucleotide or a pharmaceutical composition of the present invention is administered locally or systemically for example orally, sublingually, nasally, subcutaneously, intravenously, intraperitoneally, intramuscularly, intratumoral, intrathecal, transdermal and/or rectal. Further routes of administration include, but are not limited to, electroporation, epidermal, impression into skin, intra-arterial, intra-articular, intracranial, intradermal, intra-lesional, intra-muscular, intranasal, intra-ocular, intrathecal, intracameral, intraperitoneal, intraprostatic, intrapulmonary, intraspinal, intravesical, placement within cavities of the body, nasal pulmonary inhalation (e.g., by inhalation or insufflation of powders or aerosols, including by nebulizer), subdermal, topical (including ophthalmic and to mucous membranes including vaginal and rectal delivery), or transdermal administration. Alternatively or in combination ex vivo treated immune cells are administered. The oligonucleotide is administered alone or in combination with another oligonucleotide of the present invention and optionally in combination with another compound such as another oligonucleotide different from the present invention, an antibody or a fragment thereof such as a Fab fragment, a HERA fusion protein, a ligand trap, a nanobody, a BiTe, a small molecule and/or a chemotherapeutic (e.g., platinum, gemcitabine). In some embodiments, the other oligonucleotide (i.e., not being part of the present invention), the antibody, and/or the small molecule are effective in preventing and/or treating an autoimmune disorder, for example autoimmune arthritis or gastrointestinal autoimmune diseases such as inflammatory bowel disease (IBD) or colitis, an immune disorder, for example an immune exhaustion due to chronic viral infections such as HIV infection, a cardiovascular disorder, an inflammatory disorder for example a chronic airway inflammation, a bacterial, viral and/or fungal infection for example sepsis or a Mycobacterium bovis infection, a liver disorder, a chronic kidney disorder, a psychiatric disorder (e.g., schizophrenia, bipolar disorders, Alzheimer's disease) and/or cancer. An oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are solid tumors, blood born tumors, leukemia, tumor metastasis, hemangiomas, acoustic neuromas, neurofibroma, trachoma, pyogenic granulomas, psoriasis, astrocytoma, blastoma, Ewing's tumor, craniopharyngioma, ependymoma, medulloblastoma, glioma, hemangioblastoma, Hodgkin's lymphoma, mesothelioma, neuroblastoma, non-Hodgkin's lymphoma, pinealoma, retinoblastoma, sarcoma, seminoma, and Wilms' tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.

It is a great advantage of the present invention that an oligonucleotide of the present invention is detectable and effective in a tumor even if the oligonucleotide is administered systemically.

In some embodiments two or more oligonucleotides of the present invention are administered together, at the same time point for example in a pharmaceutical composition or separately, or on staggered intervals. In other embodiments, one or more oligonucleotides of the present invention are administered together with another compound such as another oligonucleotide (i.e., not being part of the present invention), an antibody, a small molecule and/or a chemotherapeutic, at the same time point for example in a pharmaceutical composition or separately, or on staggered intervals. In some embodiments of these combinations, the immunosuppression-reverting oligonucleotide inhibits the expression and activity, respectively, of an immune suppressive factor and the other oligonucleotide (i.e., not being part of the present invention), the antibody or a fragment thereof such as a Fab fragment, a HERA fusion protein, a ligand trap, a nanobody, a BiTe and/or small molecule inhibits (antagonist) or stimulates (agonist) the same and/or another immune suppressive factor. The immune suppressive factor and/or the immune stimulatory factor and/or an immune stimulatory factor. The immune suppressive factor is for example selected from the group consisting IDO1, IDO2, CTLA-4, PD-1, PD-L1, LAG-3, VISTA, A2AR, CD39, CD73, STAT3, TDO2, TIM-3, TIGIT, TGF-beta, BTLA, MICA, NKG2A, KIR, CD160, Chop, Xbp1 and a combination thereof. The immune stimulatory factor is for example selected from the group consisting of 4-1BB, Ox40, KIR, GITR, CD27, 2B4 and a combination thereof. The oligonucleotide and the pharmaceutical composition, respectively, is for example formulated as dosage unit in form of capsules, tablets and pills etc., respectively, which contain for example compounds selected from the group consisting of microcrystalline cellulose, gum, gelatin, starch, lactose, stearates, sweetening agent, flavouring agent and a combination thereof. For capsules the dosage unit contain for example a liquid carrier like fatty oils. Optionally, coatings of sugar or enteric agents are part of the dosage unit.

For parenteral, subcutaneous, intradermal or topical administration the oligonucleotide and/or the pharmaceutical composition include for example a sterile diluent, buffers, regulators of toxicity and antibacterials. In a preferred embodiment, the oligonucleotide or pharmaceutical composition is prepared with carriers that protect against degradation or immediate elimination from the body, including implants or microcapsules with controlled release properties. For intravenous administration carriers are for example physiological saline or phosphate buffered saline. An oligonucleotide and/or a pharmaceutical composition comprising such oligonucleotide for oral administration includes for example powder or granule, microparticulate, nanoparticulate, suspension or solution in water or non-aqueous media, capsule, gel capsule, sachet, tablet or minitablet. An oligonucleotide and/or a pharmaceutical composition comprising for parenteral, intrathecal, intracameral or intraventricular administration includes for example sterile aqueous solutions which optionally contain buffer, diluent and/or other suitable additive such as penetration enhancer, carrier compound and/or other pharmaceutically acceptable carrier or excipient.

A pharmaceutically acceptable carrier is for example liquid or solid, and is for example selected with the planned manner of administration in mind so as to provide for the desired bulk, consistency, etc., when combined with a nucleic acid and the other components of a given pharmaceutical composition. Typical pharmaceutically acceptable carriers include, but are not limited to, a binding agent (e.g., pregelatinized maize starch, polyvinylpyrrolidone or hydroxypropyl methylcellulose, etc.); filler (e.g., lactose and other sugars, microcrystalline cellulose, pectin, gelatin, calcium sulfate, ethyl cellulose, polyacrylates or calcium hydrogen phosphate, etc.); lubricant (e.g., magnesium stearate, talcum, silica, colloidal silicon dioxide, stearic acid, metallic stearates, hydrogenated vegetable oils, corn starch, polyethylene glycols, sodium benzoate, sodium acetate, etc.); disintegrate (e.g., starch, sodium starch glycolate, etc.); or wetting agent (e.g., sodium lauryl sulfate, etc.). Sustained release oral delivery systems and/or enteric coatings for orally administered dosage forms are described in U.S. Pat. Nos. 4,704,295; 4,556,552; 4,309,406; and 4,309,404. An adjuvant is included under these phrases.

The immune suppressive factor is a factor whose expression and/or activity is for example increased in a cell, tissue, organ or subject. The immune stimulatory factor is a factor whose level is increased or decreased in a cell, tissue, organ or subject depending on the cell, tissue, organ or subject and its individual conditions.

An antibody in combination with the oligonucleotide or the pharmaceutical composition of the present invention is for example an anti-PD-1 antibody, an anti-PD-L1 antibody, or a bispecific antibody. A small molecule in combination with the oligonucleotide or the pharmaceutical composition of the present invention is for example ARL67156 (Oncolmmunology 1:3; 2012) or POM-1 (Gastroenterology; 2010; 139(3): 1030-1040). A subject of the present invention is for example a mammalian, a bird or a fish. Mammals include for example horses, dogs, pigs, cats, or primates (for example, a monkey, a chimpanzee, or a lemur). Rodents include for example rats, rabbits, mice, squirrels, or guinea pigs.

### EXAMPLES

The following examples will serve to further illustrate the present invention without, at the same time, however, constituting any limitation thereof. On the contrary, it is to be clearly understood that the scope of the present invention refers to various other embodiments, modifications, and equivalents thereof which, after reading the description herein, may suggest themselves to those skilled in the art without departing from the spirit of the invention.

### Example 1: Single Dose Efficacy Screen of PD-L1 Antisense Oligonucleotides in Human Cancer Cell Lines

For the design of antisense oligonucleotides with specificity for human PD-L1 the sequence GRCh38_9_5447492_5473576 (SEQ ID NO:1) was used. 15, 16 and 17mers were designed according to in house criteria with a strong focus on specificity for the target gene. Neg1 (e.g., described in WO2014154843 A1) was used as control oligonucleotide.

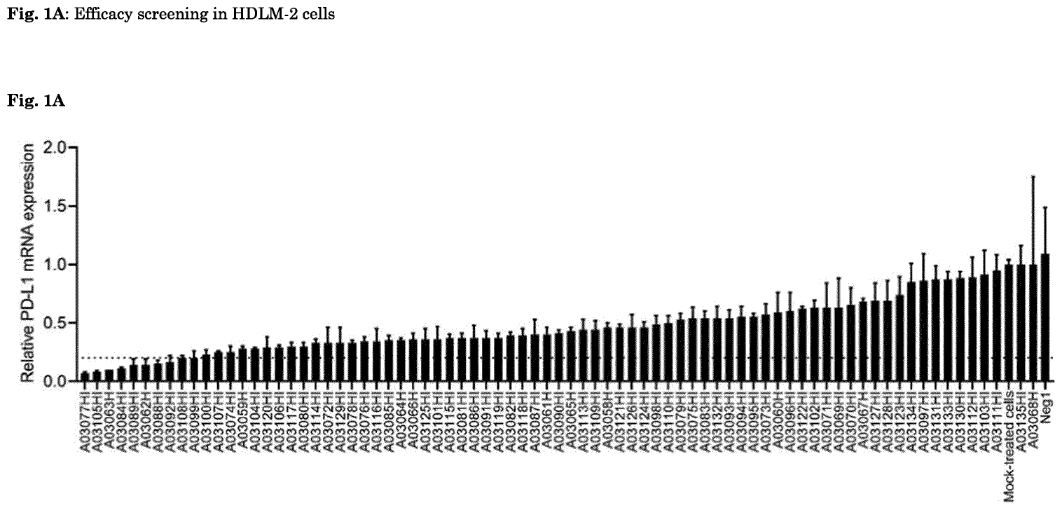

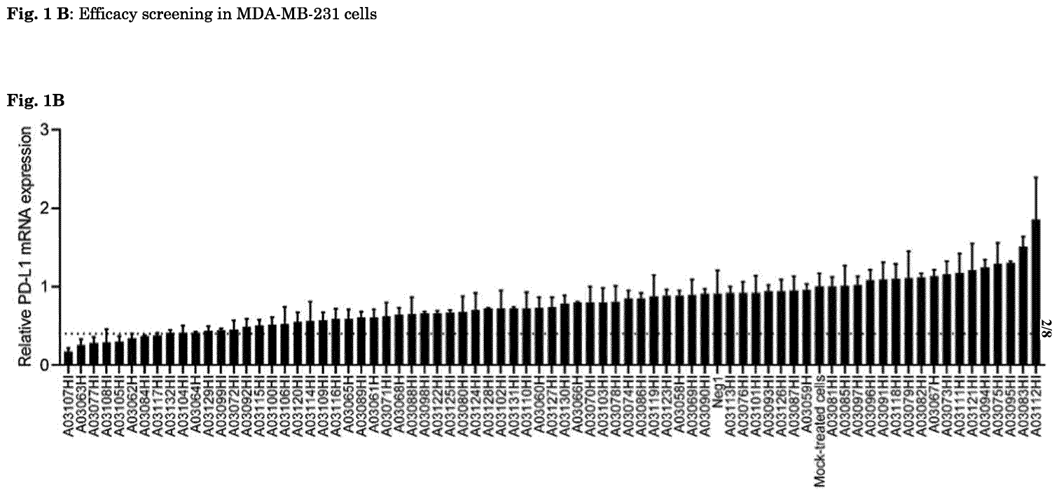

In order to investigate the knockdown efficacy of the in silico designed PD-L1 antisense oligonucleotides, efficacy screenings were performed in the human cancer cell lines HDLM-2 and MDA-MB-231. Therefore, cells were treated with the respective antisense oligonucleotides or a control oligonucleotide at a concentration of 2 μM (HDLM-2) or 10 μM (MDA-MB-231) for three days without the use of a transfection reagent. Cells were lyzed after the three days treatment period, PD-L1 and HPRT1 mRNA expression was analyzed using the QuantiGene Singleplex assay (ThermoFisher). PD-L1 expression values were normalized to HPRT1 expression values and set in relation to mock-treated cells. The results are shown in FIG. 1 and Table 1. Eight of the tested antisense oligonucleotides had a knockdown efficacy of >80% (represented by a relative PD-L1 mRNA expression of <0.2) in HDLM-2 cells (FIG. 1A). Four of the tested antisense oligonucleotides had a knockdown efficacy of >70% (represented by a relative PD-L1 mRNA expression of <0.3) in MDA-MB-231 cells (FIG. 1B).

The inhibitory effect of these oligonucleotides is shown in the following Tables 3 and 4:

TABLE 3
List of the mean PD-L1 expression values in
HDLM-2 cells as compared to mock-treated cells.
Expression values are normalized to HPRT1.

| | Residual PD-L1 expression |
|---|---|
| | in HDLM-2 cells |
| Name | (compared to mock-treated cells) |
| A03077HI | 0.07 |
| A03105HI | 0.08 |
| A03063H | 0.10 |
| A03084HI | 0.11 |
| A03089HI | 0.14 |
| A03062H | 0.14 |
| A03088HI | 0.15 |
| A03092HI | 0.16 |
| A03108HI | 0.20 |
| A03099HI | 0.20 |
| A03100HI | 0.23 |
| A03107HI | 0.25 |
| A03074HI | 0.25 |
| A03059H | 0.28 |
| A03104HI | 0.28 |
| A03120HI | 0.29 |
| A03106HI | 0.29 |
| A03117HI | 0.30 |
| A03080HI | 0.30 |
| A03114HI | 0.33 |
| A03072HI | 0.33 |
| A03129HI | 0.33 |
| A03078HI | 0.33 |
| A03076HI | 0.34 |
| A03116HI | 0.34 |
| A03085HI | 0.35 |
| A03064H | 0.35 |
| A03066H | 0.36 |
| A03125HI | 0.36 |
| A03101HI | 0.36 |
| A03115HI | 0.37 |
| A03081HI | 0.37 |
| A03086HI | 0.37 |
| A03091HI | 0.37 |
| A03119HI | 0.37 |
| A03082HI | 0.39 |
| A03118HI | 0.39 |
| A03087HI | 0.40 |
| A03061H | 0.40 |
| A03090HI | 0.41 |
| A03065H | 0.43 |
| A03113HI | 0.44 |
| A03109HI | 0.44 |
| A03058H | 0.46 |
| A03121HI | 0.46 |
| A03126HI | 0.46 |
| A03124HI | 0.46 |
| A03098HI | 0.49 |
| A03110HI | 0.50 |
| A03079HI | 0.53 |
| A03075HI | 0.54 |
| A03083HI | 0.54 |
| A03132HI | 0.54 |
| A03093HI | 0.54 |
| A03094HI | 0.55 |
| A03095HI | 0.55 |
| A03073HI | 0.57 |
| A03060H | 0.59 |
| A03096HI | 0.60 |
| A03122HI | 0.62 |
| A03102HI | 0.63 |
| A03071HI | 0.63 |
| A03069HI | 0.63 |
| A03070HI | 0.65 |
| A03067H | 0.68 |
| A03127HI | 0.69 |
| A03128HI | 0.69 |
| A03123HI | 0.74 |
| A03134HI | 0.85 |
| A03097HI | 0.86 |
| A03131HI | 0.87 |
| A03133HI | 0.87 |
| A03130HI | 0.88 |
| A03112HI | 0.89 |
| A03103HI | 0.91 |
| A03111HI | 0.95 |
| Mock-treated cells | 1.00 |
| A03135HI | 1.00 |
| A03068H | 1.00 |
| Neg1 | 1.09 |

TABLE 4
List of the mean PD-L1 expression values in
MDA-MB-231 cells as compared to mock-treated cells.
Expression values are normalized to HPRT1.

| | Residual PD-L1 expression in |
|---|---|
| | MDA-MB-231 cells |
| Name | (compared to mock-treated cells) |
| A03107HI | 0.18 |
| A03063H | 0.26 |
| A03077HI | 0.28 |
| A03108HI | 0.29 |
| A03105HI | 0.30 |
| A03062H | 0.34 |
| A03084HI | 0.37 |
| A03117HI | 0.38 |
| A03132HI | 0.41 |
| A03104HI | 0.41 |
| A03064H | 0.41 |
| A03129HI | 0.44 |
| A03099HI | 0.45 |
| A03072HI | 0.46 |
| A03092HI | 0.49 |
| A03115HI | 0.51 |
| A03100HI | 0.52 |
| A03106HI | 0.53 |
| A03120HI | 0.55 |
| A03114HI | 0.56 |
| A03109HI | 0.57 |
| A03116HI | 0.59 |
| A03065H | 0.59 |
| A03089HI | 0.61 |
| A03061H | 0.61 |
| A03071HI | 0.62 |
| A03068H | 0.64 |
| A03088HI | 0.65 |
| A03098HI | 0.66 |
| A03122HI | 0.66 |
| A03125HI | 0.67 |
| A03080HI | 0.68 |
| A03124HI | 0.70 |
| A03128HI | 0.72 |
| A03102HI | 0.72 |
| A03131HI | 0.72 |
| A03110HI | 0.72 |
| A03060H | 0.73 |
| A03127HI | 0.74 |
| A03130HI | 0.78 |
| A03066H | 0.80 |
| A03070HI | 0.80 |
| A03103HI | 0.80 |
| A03078HI | 0.81 |
| A03074HI | 0.85 |
| A03086HI | 0.85 |
| A03119HI | 0.88 |
| A03123HI | 0.89 |
| A03058H | 0.89 |
| A03069HI | 0.90 |
| A03090HI | 0.91 |
| Neg1 | 0.91 |
| A03113HI | 0.92 |
| A03076HI | 0.92 |
| A03101HI | 0.92 |
| A03093HI | 0.94 |
| A03126HI | 0.94 |
| A03087HI | 0.95 |
| A03059H | 0.96 |
| Mock-treated cells | 1.00 |
| A03081HI | 1.00 |
| A03085HI | 1.01 |
| A03097HI | 1.02 |
| A03096HI | 1.08 |
| A03091HI | 1.09 |
| A03118HI | 1.10 |
| A03079HI | 1.11 |
| A03082HI | 1.12 |
| A03067H | 1.13 |
| A03073HI | 1.16 |
| A03111HI | 1.18 |
| A03121HI | 1.21 |
| A03094HI | 1.25 |
| A03075HI | 1.29 |
| A03095HI | 1.30 |
| A03083HI | 1.51 |
| A03112HI | 1.85 |

### Example 2: Investigation of the Dose-Response Relationship and Determination of IC₅₀ Values of Selected PD-L1 Antisense Oligonucleotides

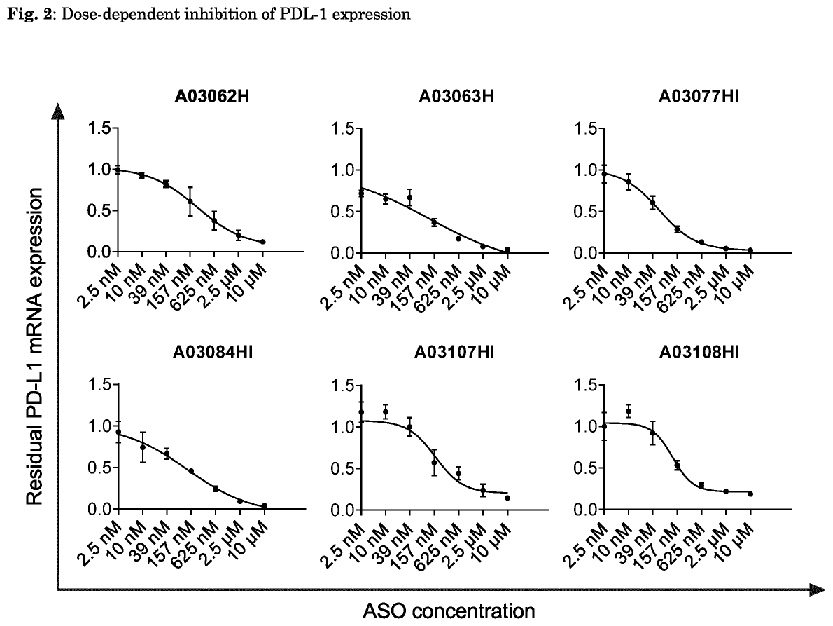

The dose-dependent knockdown of PD-L1 mRNA expression by PD-L1 antisense oligonucleotides was investigated in HDLM-2 cells and the respective ID₅₀ values were calculated. Therefore, HDLM-2 cells were treated at the following concentrations: 10 PC 2.5 μM, 625 nM, 157 nM, 39 nM, 10 nM, 2.5 nM of the respective antisense oligonucleotide for three days without the use of a transfection reagent. Cells were lyzed after the three days treatment period, PD-L1 and HPRT1 mRNA expression was analyzed using the QuantiGene Singleplex assay (ThermoFisher). PD-L1 expression values were normalized to HPRT1 expression values and set in relation to mock-treated cells. A dose-dependent knockdown of PD-L1 mRNA with all tested PD-L1 antisense oligonucleotides was observed (FIG. 2, Table 5) with IC₅₀ values in the nanomolar range shown in the following Table 5:

TABLE 5
IC₅₀ values and knockdown efficacy of selected PD-L1 antisense oligonucleotides.

| | | Knockdown efficacy (%) | | | | | | |
|---|---|---|---|---|---|---|---|---|
| ASO | IC50 | 2.5 nM | 10 nM | 39 nM | 157 nM | 625 nM | 2.5 μM | 10 μM |
| A03062H | 234 | 0.53 | 7.42 | 17.95 | 39.06 | 62.32 | 80.28 | 88.05 |
| A03063H | 116 | 28.13 | 34.86 | 33.02 | 63.14 | 82.75 | 92.14 | 95.65 |
| A03077HI | 56 | 4.81 | 14.42 | 39.40 | 71.49 | 86.62 | 94.62 | 96.68 |
| A03084HI | 128 | 7.26 | 25.59 | 33.44 | 54.14 | 75.57 | 90.52 | 95.80 |
| A03107HI | 168 | 0.00 | 0.00 | 0.00 | 42.93 | 55.86 | 76.20 | 85.36 |
| A03108HI | 122 | 0.00 | 0.00 | 8.01 | 46.73 | 71.36 | 78.04 | 81.21 |

### Example 3: In Vivo Assessment of Liver Toxicity of Selected Antisense Oligonucleotides

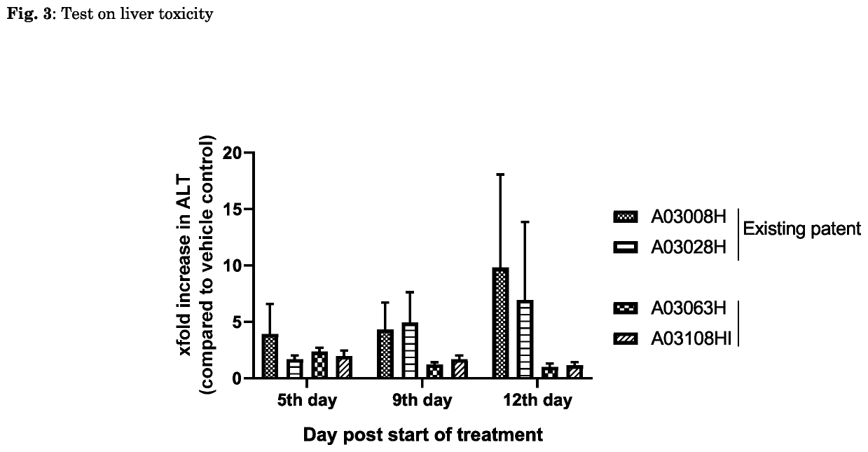

In order to determine the liver toxic capacity of the antisense oligonucleotides A03008H and A03028 (described in WO 2018/065589 A1), and of the antisense oligonucleotides A03063H (SEQ ID NO.7) and A03108HI (SEQ ID NO.50) of the present invention mice were treated with repeated injections (20 mg/kg) of the respective antisense oligonucleotide. The serum levels of Alanine transaminase were determined at different time points. As shown in FIG. 3, treatment with two both tested antisense oligonucleotides of WO 2018/065589 A1 led to an increase in ALT and some of the treated mice had to be sacrificed. In contrast, treatment with none of the two tested antisense oligonucleotides led to an increase in ALT as compared to the vehicle control.

### Example 4: Investigation of Potential Off-Target Binding Sites

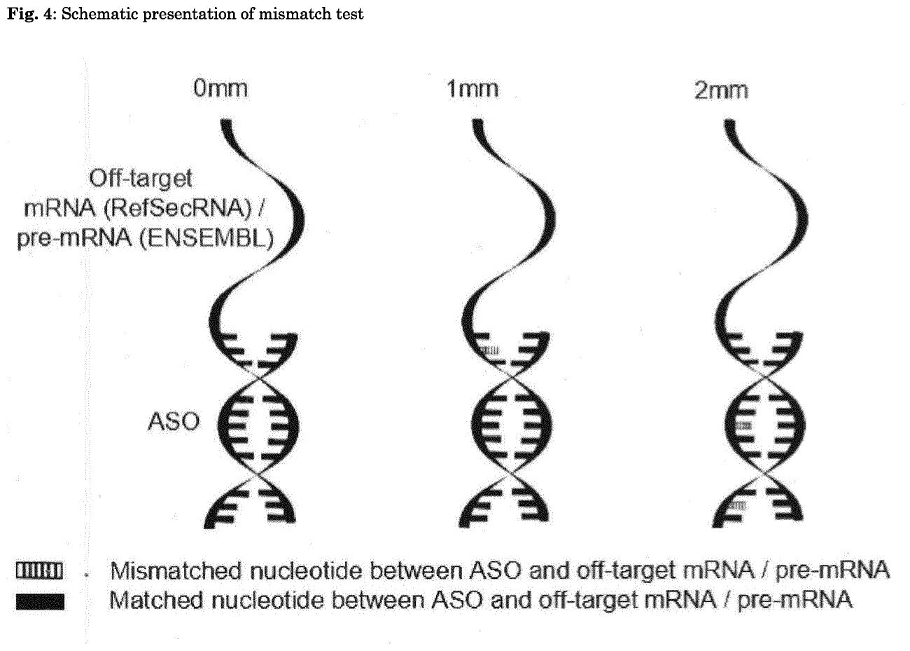

Two different databases were screened in silico to test off-target effects of oligonucleotides of the present invention. These databases were RefSecRNA comprising sequences of spliced RNA and ENSEMBL comprising sequences of non-spliced RNA. The oligonucleotides shown in Tables 6 and 7 have no potential off-target binding site with zero mismatches, i.e., 100% sequence complementarity (0 mm) to an off-target sequence or one mismatch, i.e., ((n−1)/n*100) % sequence complementarity (1 mm) to an off-target sequence. The number of potential off-target sites of an oligonucleotide of the present invention having two mismatches, i.e., ((n−2)/n*100) % sequence complementarity (2 mm) is limited to max. 20 (see Tables 6 and 7, RefSeq (Gene Ids), 2 mm). FIG. 4 shows the principle of the mismatch test.

Tables 6 and 7 depicts the results of the mismatch test for the oligonucleotides of the present invention.

TABLE 6
Number of genes, besides the target gene, that show a sequence
complementarity with the respective PD-L1 exon-binding
antisense oligonucleotide allowing 0, 1 or 2 mismatches.

| | RefSeq (Gene Ids) | | | ENSEMBL | | |
|---|---|---|---|---|---|---|
| Name | 0 mm | 1 mm | 2 mm | 0 mm | 1 mm | 2 mm |
| A03058H | 0 | 0 | 7 | 0 | 0 | 136 |
| A03059H | 0 | 0 | 7 | 0 | 0 | 201 |
| A03060H | 0 | 0 | 3 | 0 | 0 | 214 |
| A03061H | 0 | 0 | 10 | 0 | 0 | 106 |
| A03062H | 0 | 0 | 4 | 0 | 0 | 84 |
| A03063H | 0 | 0 | 6 | 0 | 0 | 51 |
| A03064H | 0 | 0 | 6 | 0 | 0 | 60 |
| A03065H | 0 | 0 | 4 | 0 | 0 | 71 |
| A03066H | 0 | 0 | 7 | 0 | 0 | 68 |
| A03067H | 0 | 0 | 20 | 0 | 0 | 130 |
| A03068H | 0 | 0 | 13 | 0 | 0 | 283 |

TABLE 7
Number of genes, besides the target gene, that show a
sequence complementarity with the respective RD-L1
intron-binding antisense oligonucleotide allowing
0, 1 or 2 mismatches.

| | RefSeq (Gene Ids) | | | ENSEMBL | | |
|---|---|---|---|---|---|---|
| Name | 0 mm | 1 mm | 2 mm | 0 mm | 1 mm | 2 mm |
| A03069HI | 0 | 0 | 2 | 0 | 0 | 32 |
| A03070HI | 0 | 0 | 11 | 0 | 0 | 97 |
| A03071HI | 0 | 0 | 3 | 0 | 0 | 20 |
| A03072HI | 0 | 0 | 10 | 0 | 0 | 76 |
| A03073HI | 0 | 0 | 1 | 0 | 0 | 75 |
| A03074HI | 0 | 0 | 8 | 0 | 0 | 34 |
| A03075HI | 0 | 0 | 7 | 0 | 0 | 50 |
| A03076HI | 0 | 0 | 5 | 0 | 0 | 57 |
| A03077HI | 0 | 0 | 6 | 0 | 0 | 44 |
| A03078HI | 0 | 0 | 3 | 0 | 0 | 25 |
| A03079HI | 0 | 0 | 3 | 0 | 0 | 25 |
| A03080HI | 0 | 0 | 9 | 0 | 0 | 84 |
| A03081HI | 0 | 0 | 3 | 0 | 0 | 40 |
| A03082HI | 0 | 0 | 1 | 0 | 0 | 22 |
| A03083HI | 0 | 0 | 3 | 0 | 0 | 57 |
| A03084HI | 0 | 0 | 7 | 0 | 0 | 54 |
| A03085HI | 0 | 0 | 1 | 0 | 0 | 10 |
| A03086HI | 0 | 0 | 1 | 0 | 0 | 39 |
| A03087HI | 0 | 0 | 4 | 0 | 0 | 37 |
| A03088HI | 0 | 0 | 8 | 0 | 0 | 30 |
| A03089HI | 0 | 0 | 0 | 0 | 0 | 28 |
| A03090HI | 0 | 0 | 7 | 0 | 0 | 62 |
| A03091HI | 0 | 0 | 3 | 0 | 0 | 31 |
| A03092HI | 0 | 0 | 7 | 0 | 0 | 43 |
| A03093HI | 0 | 0 | 5 | 0 | 0 | 96 |
| A03094HI | 0 | 0 | 4 | 0 | 0 | 57 |
| A03095HI | 0 | 0 | 3 | 0 | 0 | 43 |
| A03096HI | 0 | 0 | 8 | 0 | 0 | 28 |
| A03097HI | 0 | 0 | 3 | 0 | 0 | 34 |
| A03098HI | 0 | 0 | 4 | 0 | 0 | 28 |
| A03099HI | 0 | 0 | 2 | 0 | 0 | 58 |
| A03100HI | 0 | 0 | 3 | 0 | 0 | 43 |
| A03101HI | 0 | 0 | 4 | 0 | 0 | 31 |
| A03102HI | 0 | 0 | 2 | 0 | 0 | 48 |
| A03103HI | 0 | 0 | 2 | 0 | 0 | 48 |
| A03104HI | 0 | 0 | 9 | 0 | 0 | 46 |
| A03105HI | 0 | 0 | 9 | 0 | 0 | 84 |
| A03106HI | 0 | 0 | 6 | 0 | 0 | 68 |
| A03107HI | 0 | 0 | 4 | 0 | 0 | 85 |
| A03108HI | 0 | 0 | 2 | 0 | 0 | 26 |
| A03109HI | 0 | 0 | 1 | 0 | 0 | 33 |
| A03110HI | 0 | 0 | 4 | 0 | 0 | 35 |
| A03111HI | 0 | 0 | 6 | 0 | 0 | 59 |
| A03112HI | 0 | 0 | 2 | 0 | 0 | 29 |
| A03113HI | 0 | 0 | 8 | 0 | 0 | 66 |
| A03114HI | 0 | 0 | 0 | 0 | 0 | 19 |
| A03115HI | 0 | 0 | 7 | 0 | 0 | 25 |
| A03116HI | 0 | 0 | 7 | 0 | 0 | 79 |
| A03117HI | 0 | 0 | 0 | 0 | 0 | 26 |
| A03118HI | 0 | 0 | 5 | 0 | 0 | 35 |
| A03119HI | 0 | 0 | 3 | 0 | 0 | 31 |
| A03120HI | 0 | 0 | 2 | 0 | 0 | 26 |
| A03121HI | 0 | 0 | 2 | 0 | 0 | 30 |
| A03122HI | 0 | 0 | 10 | 0 | 0 | 153 |
| A03123HI | 0 | 0 | 10 | 0 | 0 | 134 |
| A03124HI | 0 | 0 | 2 | 0 | 0 | 39 |
| A03125HI | 0 | 0 | 8 | 0 | 0 | 97 |
| A03126HI | 0 | 0 | 9 | 0 | 0 | 64 |
| A03127HI | 0 | 0 | 7 | 0 | 0 | 93 |
| A03128HI | 0 | 0 | 8 | 0 | 0 | 57 |
| A03129HI | 0 | 0 | 6 | 0 | 0 | 80 |
| A03130HI | 0 | 0 | 4 | 0 | 0 | 34 |
| A03131HI | 0 | 0 | 2 | 0 | 0 | 44 |
| A03132HI | 0 | 0 | 3 | 0 | 0 | 87 |
| A03133HI | 0 | 0 | 9 | 0 | 0 | 31 |
| A03134HI | 0 | 0 | 9 | 0 | 0 | 84 |
| A03135HI | 0 | 0 | 9 | 0 | 0 | 84 |

### Example 5: Dose-Dependent PD-L1 Protein Knockdown and IC₅₀ Values in Two Different Cell Lines

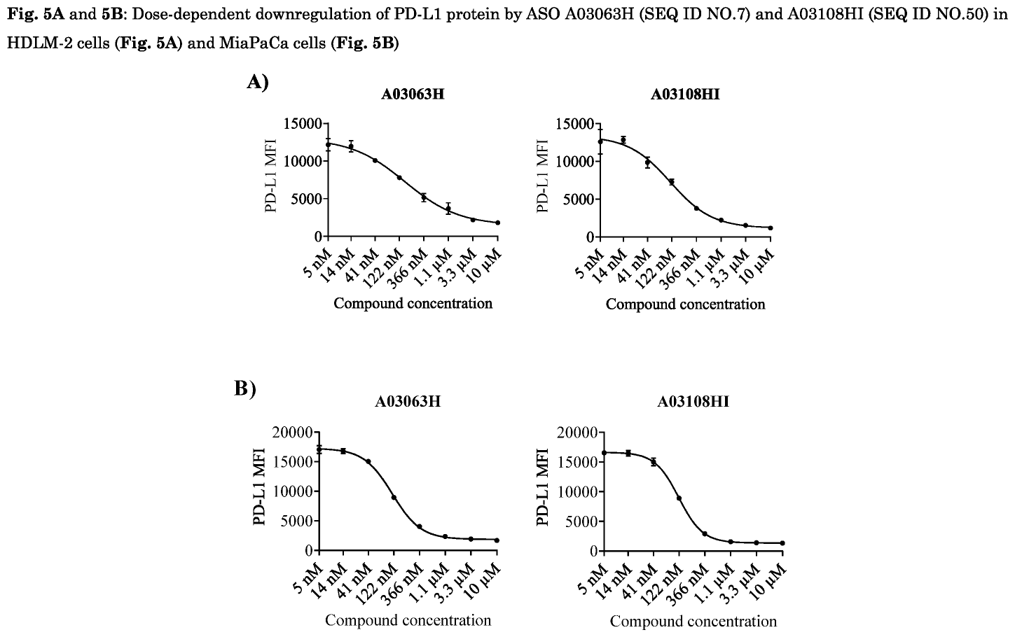

Two different cancer cell lines were used for investigation of PD-L1 protein knockdown efficacy of the ASOs A03063H (SEQ ID NO.7) and A03108HI (SEQ ID NO.50). Therefore, HDLM-2 cells (human Hodgkin lymphoma cells) or MiaPaCa-2 cells (human pancreas carcinoma cells) were treated with the indicated ASO at different concentrations for three days without the use of a transfection reagent (gymnotic transfection). In order to induce expression of PD-L1 in MiaPaCa-2 cells IFN gamma was added to the cells two days after start of treatment with the ASO. On day three after start of ASO treatment PD-L1 protein expression was assessed by flow cytometry using a PD-L1-specific antibody. The median fluorescence intensity (MFI) of PD-L1 as a measure of protein expression is shown for HDLM-2 (FIG. 5A) and MiaPaCa-2 cells (FIG. 5B) after treatment of cells with the respective ASO at the respective concentration. Table 8 (HDLM-2 cells) and Table 9 (MiaPaCa-2 cells) show IC₅₀ values and knockdown efficacy at the respective concentrations.

TABLE 8
IC₅₀ values of ASOs A03063H (SEQ ID NO. 7) and A03108HI (SEQ ID
NO. 50) in HDLM-2 cells.

| | IC₅₀ | % Inhibition | | | | | | | |
|---|---|---|---|---|---|---|---|---|---|
| | (nM) | 5 nM | 14 nM | 41 nM | 122 nM | 366 nM | 1.1 μM | 3.3 μM | 10 μM |
| A03063H | 157 | 19 | 21 | 33 | 48 | 66 | 76 | 85 | 88 |
| A03108HI | 143 | 17 | 15 | 35 | 52 | 75 | 85 | 90 | 92 |

TABLE 9
IC₅₀ values of ASOs A03063H (SEQ ID NO. 7) and A03108HI (SEQ ID
NO. 50) in MiaPaCa-2 cells.

| | IC₅₀ | % Inhibition | | | | | | | |
|---|---|---|---|---|---|---|---|---|---|
| | (nM) | 5 nM | 14 nM | 41 nM | 122 nM | 366 nM | 1.1 μM | 3.3 μM | 10 μM |
| A03063H | 114 | 6 | 8 | 17 | 51 | 78 | 87 | 89 | 91 |
| A03108HI | 121 | 9 | 10 | 18 | 51 | 84 | 91 | 92 | 93 |

### Example 6: PD-L1 Protein Knockdown in Dendritic Cells

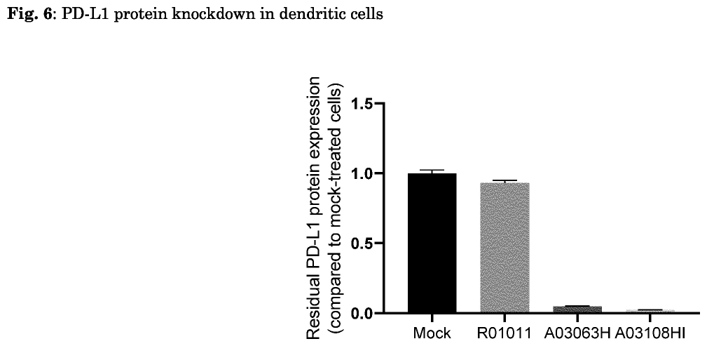

Here the knockdown efficacy of PD-L1 ASOs A03063H (SEQ ID NO.7) and A03108HI (SEQ ID NO.50) was investigated in human dendritic cells (DC). Therefore, DC were generated in a three day protocol: human monocytes were purified out of peripheral blood mononuclear cells (PBMC), differentiated to immature DC by addition of interleukin-4 (IL-4) and granulocyte-macrophage colony stimulating factor (GM-CSF) and matured into DC by addition of a cytokine cocktail and a toll-like receptor ligand. Cells were either not treated with ASO (Mock), treated with a control oligonucleotide (R01011, SEQ ID NO.78) or one of the PD-L1-specific ASOs A03063H (SEQ ID NO.7) or A03108HI (SEQ ID NO.50) at a final concentration of 5 μM during the generation of DC. As shown in FIG. 6, R01011 had only little impact on residual PD-L1 protein expression as compared to mock-treated cells. In strong contrast, treatment with A03063H or A03108HI led to a >90% reduction of PD-L1 protein expression (represented by a residual PD-L1 protein expression of <0.1)

### Example 7: Persistency of PD-L1 Protein Knockdown in Highly Proliferating Cells

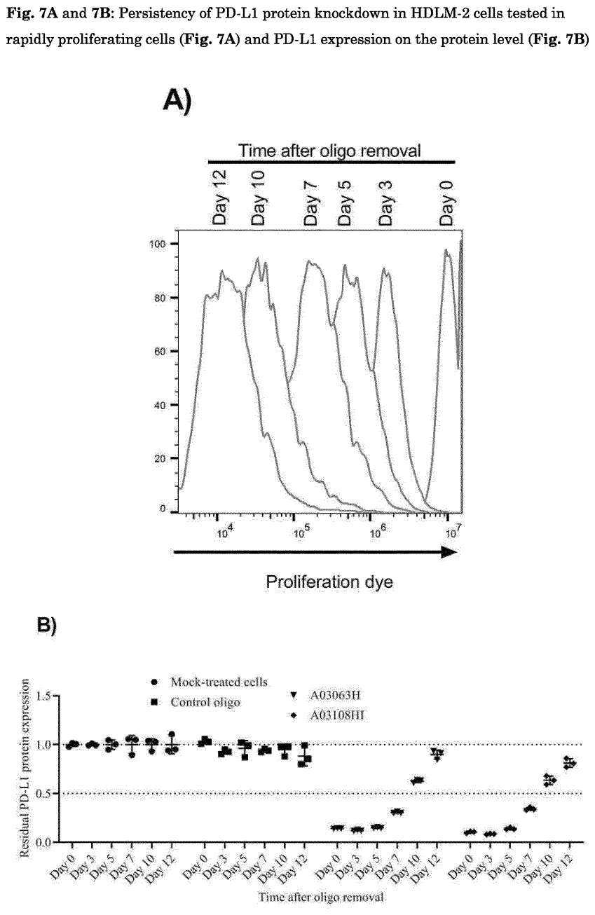

Further, the persistency of PD-L1 protein knockdown in PD-L1 ASO-treated, highly proliferating HDLM-2 cells was investigated. Therefore, HDLM-2 cells (human Hodgkin lymphoma cells) were either not treated (Mock-treated cells), treated with the control oligo neg1 (SEQ ID NO.77) or the PD-L1-specific ASOs A03063H (SEQ ID NO.7) or A03108HI (SEQ ID NO.50) at a final concentration of 5 μM. Three days later, cells were stringently washed in order to remove the ASO and stained with a cell proliferation dye. Cells were analyzed by flow cytometry on day 0, 3, 5, 7, 10 and 12 after washing away the ASO with regard to proliferation (FIG. 7A) and PD-L1 expression (FIG. 7B, shown as residual PD-L1 protein expression as compared to mock-treated cells from the same day). As shown by homogenous dilution of the proliferation dye over time in FIG. 7A, homogenous, strong proliferation of HDLM-2 cells was observed on day 3, 5, 7, 10 and 12 after washing away the ASO. A negative impact of neg1 on PD-L1 protein expression was not observed. In strong contrast, treatment of cells with the PD-L1-specific ASOs A03063H or A03108HI led to a strong and persistent reduction of PD-L1 protein expression with an efficacy of >50% up to day 7 after washing away the ASO.

The results surprisingly show that despite intensive proliferation of the cells and thus, quick dilution of the ASO a strong inhibition of the PD-L1 protein expression was detectable for numerous days, i.e., up to 7 days.

1. Oligonucleotide consisting of 10 to 20 nucleotides hybridizing with SEQ ID NO.1 encoding PD-L1, wherein the oligonucleotide hybridizes within the region of from position 15400 to position 22850 of SEQ ID NO.1 or within the region of from position 3100 to position 19500 of SEQ ID NO.1.

2. Oligonucleotide according to claim 1, wherein the oligonucleotide does only hybridize with the target RNA with zero mismatches.

3. Oligonucleotide according to claim 1, wherein the oligonucleotide has at least two mismatches to an off-target nucleotide sequence and has max. 20 off-target bindings having two mismatches.

4. Oligonucleotide according to claim 1, wherein the oligonucleotide comprises one or more modified nucleotides.

5. Oligonucleotide according to claim 1, wherein the oligonucleotide comprises a LNA, a c-ET, an ENA, a polyalkylene oxide-, a 2′-fluoro-, a 2′-O-methoxy-, a FANA and/or a 2′-O-methyl-modified nucleotide.

6. Oligonucleotide according to claim 1, wherein the modified nucleotide(s) is/are located at the 5′- or 3′-end, or at the 5′- and 3′-end of the oligonucleotide.

7. Oligonucleotide according to claim 1, wherein the oligonucleotide comprises a sequence selected from the group consisting of SEQ ID NO.7, SEQ ID NO.2, SEQ ID NO.3, SEQ ID NO.4, SEQ ID NO.5, SEQ ID NO.6, SEQ ID NO.8, SEQ ID NO.9, SEQ ID NO.10, SEQ ID NO.11, SEQ ID NO.12 and a combination thereof.

8. Oligonucleotide according to wherein the oligonucleotide comprises a sequence selected from the group consisting of SEQ ID NO.50, SEQ ID NO.13, SEQ ID NO.14, SEQ ID NO.15, SEQ ID NO.16, SEQ ID NO.17, SEQ ID NO.18, SEQ ID NO.19, SEQ ID NO.20, SEQ ID NO.21, SEQ ID NO.22, SEQ ID NO.23, SEQ ID NO.24, SEQ ID NO.25, SEQ ID NO.26, SEQ ID NO.27, SEQ ID NO.28, SEQ ID NO.29, SEQ ID NO.30, SEQ ID NO.31, SEQ ID NO.32, SEQ ID NO.33, SEQ ID NO.34, SEQ ID NO.35, SEQ ID NO.36, SEQ ID NO.37, SEQ ID NO.38, SEQ ID NO.39, SEQ ID NO.40, SEQ ID NO.41, SEQ ID NO.42, SEQ ID NO.43, SEQ ID NO.44, SEQ ID NO.45, SEQ ID NO.46, SEQ ID NO.47, SEQ ID NO.48, SEQ ID NO.49, SEQ ID NO.51, SEQ ID NO.52, SEQ ID NO.53, SEQ ID NO.54, SEQ ID NO.55, SEQ ID NO.56, SEQ ID NO.57, SEQ ID NO.58, SEQ ID NO.59, SEQ ID NO.60, SEQ ID NO.61, SEQ ID NO.62, SEQ ID NO.63, SEQ ID NO.64, SEQ ID NO.65, SEQ ID NO.66, SEQ ID NO.67, SEQ ID NO.68, SEQ ID NO.69, SEQ ID NO.70, SEQ ID NO.71, SEQ ID NO.72, SEQ ID NO.73, SEQ ID NO.74, SEQ ID NO.75, SEQ ID NO.76 and a combination thereof.

9. Oligonucleotide according to claim 1, wherein the oligonucleotide comprises a sequence selected from the group consisting of SEQ ID NO.7, SEQ ID NO.50, SEQ ID NO.2, SEQ ID NO.3, SEQ ID NO.4, SEQ ID NO.5, SEQ ID NO.6, SEQ ID NO.8, SEQ ID NO.9, SEQ ID NO.10, SEQ ID NO.11, SEQ ID NO.12, SEQ ID NO.13, SEQ ID NO.14, SEQ ID NO.15, SEQ ID NO.16, SEQ ID NO.17, SEQ ID NO.18, SEQ ID NO.19, SEQ ID NO.20, SEQ ID NO.21, SEQ ID NO.22, SEQ ID NO.23, SEQ ID NO.24, SEQ ID NO.25, SEQ ID NO.26, SEQ ID NO.27, SEQ ID NO.28, SEQ ID NO.29, SEQ ID NO.30, SEQ ID NO.31, SEQ ID NO.32, SEQ ID NO.33, SEQ ID NO.34, SEQ ID NO.35, SEQ ID NO.36, SEQ ID NO.37, SEQ ID NO.38, SEQ ID NO.39, SEQ ID NO.40, SEQ ID NO.41, SEQ ID NO.42, SEQ ID NO.43, SEQ ID NO.44, SEQ ID NO.45, SEQ ID NO.46, SEQ ID NO.47, SEQ ID NO.48, SEQ ID NO.49, SEQ ID NO.51, SEQ ID NO.52, SEQ ID NO.53, SEQ ID NO.54, SEQ ID NO.55, SEQ ID NO.56, SEQ ID NO.57, SEQ ID NO.58, SEQ ID NO.59, SEQ ID NO.60, SEQ ID NO.61, SEQ ID NO.62, SEQ ID NO.63, SEQ ID NO.64, SEQ ID NO.65, SEQ ID NO.66, SEQ ID NO.67, SEQ ID NO.68, SEQ ID NO.69, SEQ ID NO.70, SEQ ID NO.71, SEQ ID NO.72, SEQ ID NO.73, SEQ ID NO.74, SEQ ID NO.75, SEQ ID NO.76 and a combination thereof.

10. Oligonucleotide according to claim 1, wherein the oligonucleotide is selected from the group consisting of +G*+T*+T*T*A*G*T*T*T*G*G*C*G*A*C*+A*+A (A03063H), +G*+T*+T*A*G*G*T*T*C*A*G*C*G*A*+T*+T*+A (A3108HI), +T*+T*+A*C*C*A*C*T*C*A*G*G*A*C*+T*+T*+G (A03058H), +A*+T*+G*C*T*G*G*A*T*T*A*C*G*T*+C*+T*+C (A03059H), +A*+A*+T*G*C*T*G*G*A*T*T*A*C*G*+T*+C*+T (A03060H), +A*+T*G*+A*T*T*T*G*C*T*T*G*G*+A*G*G*+C (A03061H), +G*+G*+C*G*A*C*A*A*A*A*T*T*G*T*+A*+A*+C (A03062H), +C*+A*+C*A*A*C*G*A*A*T*G*A*G*G*+C*+T*+T (A03064H), +T*+A*+G*A*C*T*A*T*G*T*G*C*C*T*+T*+G*+C (A03065H), +T*+G*+A*G*T*A*G*A*C*T*A*T*G*T*+G*+C*+C (A03066H), +T*+G*+G*A*T*T*A*C*G*T*C*T*C*+C*+T*+C (A03067H), +T*+G*+C*T*G*G*A*T*T*A*C*G*T*+C*+T*+C (A03068H), +C*+G*+A*G*C*T*A*G*C*C*A*G*A*G*+A*+T*+A (A03069HI), +C*+T*+A*G*A*C*C*A*T*C*G*C*+G*+T*+T (A03070HI), +A*+A*+T*C*G*C*G*C*C*T*G*G*A*G*+G*+A*+A (A03071HI), +A*+C*+T*G*A*A*T*C*G*C*G*C*C*+T*+G*+G (A03072HI), +T*+A*+C*C*T*A*T*C*C*T*A*T*A*C*+T*+A*+C (A03073HI), +T*+G*+G*A*C*C*T*G*C*T*T*A*G*C*+G*+C*+A (A03074HI), +A*+C*C*G*G*T*T*A*A*A*C*T*T*C*C*+T*+T (A03075HI), +T*+A*+T*G*G*C*C*T*A*C*T*C*T*G*+G*+T*+G (A03076HI), +G*+G*+A*A*T*A*G*C*G*A*T*G*G*C*+A*+T*+T (A03077HI), +T*+C*+C*G*T*C*T*A*T*C*C*T*G*T*+A*+G*+A (A03078HI), +T*+C*C*G*T*C*T*A*T*C*C*T*G*T*A*+G*+A (A03079HI), +T*+C*+C*A*A*A*C*T*G*A*C*G*T*A*+G*+A*+A (A03080HI), +C*+A*+C*C*T*T*A*C*C*A*A*A*C*C*+G*+T*+A (A03081HI), +A*+T*+C*G*T*A*A*A*T*G*C*G*G*A*+T*+G*+T (A03082HI), +T*+C*+C*T*A*T*T*A*C*A*A*T*C*G*+T*+A*+A (A03083HI), +G*+A*+G*C*T*T*G*A*C*C*A*C*A*A*+T*+T*+G (A03084HI), +A*+T*+C*G*A*T*G*C*C*A*C*G*T*A*+T*+A*+T (A03085HI), +A*+C*+T*A*A*T*C*G*A*T*G*C*C*+A*+C*+G (A03086HI), +T*+C*+A*A*C*T*A*A*T*C*G*A*T*G*+C*+C*+A (A03087HI), +T*+A*+G*T*T*A*C*A*G*G*C*C*G*T*+G*+A*+A (A03088HI), +T*+A*+T*A*G*T*T*A*C*A*G*G*C*C*+G*+T*+G (A03089HI), +T*+C*+A*T*T*G*C*G*T*A*A*A*G*T*+A*+G*+A (A03090HI), +T*+A*+T*C*T*G*G*T*C*G*G*T*T*A*+T*+G*+T (A03091HI), +A*+T*+C*A*C*T*T*T*A*T*C*T*G*G*+T*+C*+G (A03092HI), +C*+A*+C*A*G*C*G*T*T*T*A*T*A*A*+A*+T*+C (A03093HI), +A*+T*+T*G*G*C*A*C*A*G*C*G*T*T*+T*+A*+T (A03094HI), +A*+C*+T*G*A*C*G*G*A*C*C*T*A*A*+T*+A*+A (A03095HI), +C*+C*+G*A*G*G*A*A*C*T*A*A*C*A*+C*+T*+C (A03096HI), +T*+A*+C*C*G*A*G*G*A*A*C*T*A*A*+C*+A*+C (A03097HI), +G*+G*+T*C*A*A*T*A*C*C*G*A*G*G*+A*+A*+C (A03098HI), +A*+C*+G*C*C*A*T*T*G*C*A*G*G*A*+A*+A*+T (A03099HI), +G*+A*+T*T*G*A*T*G*G*T*A*G*T*T*+A*+G*+C (A3100HI), +G*+C*+C*T*G*A*T*A*T*T*T*G*C*G*G*+A*+T (A3101HI), +A*+T*+C*A*G*T*G*C*C*G*G*A*A*G*+A*+T*+T (A3102HI), +A*+T*C*A*G*T*G*C*C*G*G*A*A*G*+A*+T*+T (A3103HI), +A*+C*+C*A*T*C*A*G*T*G*C*C*G*G*+A*+A*+G (A3104HI), +G*+G*+T*T*G*C*C*T*T*G*T*T*C*T*+A*+A*+G (A3105HI), +T*+T*+G*C*A*T*A*T*G*G*A*G*G*T*+G*+A*+C (A3106HI), +A*+A*+G*C*T*A*T*G*T*T*A*C*C*A*+C*+A*+C (A3107HI), +T*+G*+T*T*A*G*G*T*T*C*A*G*C*G*+A*+T*+T (A3109HI), +A*+C*+A*G*C*A*G*T*C*G*A*T*T*T*+G*+G*+T (A3110HI), +G*+A*+A*T*G*A*C*A*G*C*A*G*T*C*+G*+A*+T (A3111HI), +A*+C*+T*C*G*A*T*A*G*T*A*G*C*A*+G*+T*+A (A3112HI), +A*+G*+T*A*C*T*C*G*A*T*A*G*T*+A*+G*+C (A3113HI), +T*+A*+G*T*A*G*T*A*C*T*C*G*A*T*+A*+G*+T (A3114HI), +T*+T*+G*T*A*G*T*A*G*T*A*C*T*C*+G*+A*+T (A3115HI), +A*+G*+T*G*C*T*A*A*T*T*G*T*A*G*+T*+A*+G (A3116HI), +A*+T*+A*C*G*T*A*C*A*C*C*A*G*A*+G*+G*+T (A3117HI), +A*+G*+A*C*C*T*C*G*C*A*G*T*G*T*+T*+A*+T (A3118HI), +A*+T*+T*A*G*A*C*C*T*C*G*C*A*G*+T*+G*+T (A3119HI), +T*+A*+C*T*T*A*A*T*T*A*G*A*C*C*+T*+C*+G (A3120HI), +T*+A*+T*C*G*G*C*C*A*C*T*G*T*A*T*+G*+A (A3121HI), +G*+A*+T*T*A*A*G*A*T*A*C*G*T*+A*+G*+T (A3122HI), +C*+A*+T*A*A*C*T*A*A*G*G*A*C*+G*+T*+T (A3123HI), +A*+T*+C*G*T*C*A*T*A*A*C*T*A*A*+G*+G*+A (A3124HI), +T*+A*+T*G*T*T*C*C*T*G*G*T*G*A*+T*+A*+C (A3125HI), +C*+A*+T*G*G*T*G*T*T*G*G*A*T*T*+G*+C*+C (A3126HI), +C*+T*+G*T*T*G*C*T*A*A*T*C*T*G*+A*+C*+C (A3127HI), +A*+C*+A*C*C*G*T*C*C*T*G*G*A*T*+T*+A*+T (A3128HI), +T*+C*+T*G*T*T*C*A*C*A*A*C*A*C*+C*+G*+T (A3129HI), +A*+A*+T*A*C*C*T*G*A*G*G*A*C*T*+C*+G*+T (A3130HI), +C*+T*+A*G*T*A*G*C*C*T*A*C*A*G*+T*+A*+C (A3131HI), +G*+C*+T*T*G*C*A*C*A*G*T*A*C*C*+A*+C*+A (A3132HI), +C*+T*+G*G*A*A*T*G*G*C*G*A*G*A*+T*+A*+C (A3133HI), +T*+C*+A*G*A*C*G*G*T*G*G*A*G*G*+A*+G*+T (A3134HI), +T*+C*+A*G*A*C*G*G*T*G*G*A*G*G*A*+G*+T (A3135HI) and a combination thereof, wherein + indicates a LNA-modified nucleotide and * indicates phosphorothioate.

11. Pharmaceutical composition comprising the oligonucleotide according to claim 1 and a pharmaceutically acceptable excipient.

12. Method of preventing and/or treating a disease or disorder selected from the list of a malignant tumor, and a benign tumor comprising administering the oligonucleotide according to claim 1 to a subject in need thereof.

13. The method of claim 12, wherein the tumor is selected from the group consisting of solid tumors, blood born tumors, leukemias, tumor metastasis, hemangiomas, acoustic neuromas, neurofibromas, trachomas, pyogenic granulomas, psoriasis, astrocytoma, blastoma, Ewing's tumor, craniopharyngioma, ependymoma, medulloblastoma, glioma, hemangioblastoma, Hodgkin's lymphoma, mesothelioma, neuroblastoma, non-Hodgkin's lymphoma, pinealoma, retinoblastoma, sarcoma, seminoma, and Wilms' tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.

14. Method of preventing and/or treating a disease or disorder selected from the group consisting of a malignant tumor and a benign tumor, comprising administering the pharmaceutical composition according to claim 11 to a subject in need thereof.

15. The method of claim 14, wherein the tumor is selected from the group consisting of solid tumors, blood born tumors, leukemias, tumor metastasis, hemangiomas, acoustic neuromas, neurofibromas, trachomas, pyogenic granulomas, psoriasis, astrocytoma, blastoma, Ewing's tumor, craniopharyngioma, ependymoma, medulloblastoma, glioma, hemangioblastoma, Hodgkin's lymphoma, mesothelioma, neuroblastoma, non-Hodgkin's lymphoma, pinealoma, retinoblastoma, sarcoma, seminoma, and Wilms' tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, and uterine cancer.


SEQUENCE LISTING (78 sequences)

SEQ ID NO: 1 - DNA, 26085 bp, Homo sapiens
tctctggtcacccatacatgttaattacccctgtcatttaaaccattgttttgtgtaactagaacaaagtattctttccacgaagtctgtttgacacgagacacatataggatgtgagtctatgacaagggtaggggcaacaaatgtgaggggttggatggaaacagaggaagagaaatgagcattgcatagggcagtggtttaaaaccctacatgatcaaaaatagaacctggagacaaatcttgatattgtctttatacataataaaatgcattttaaaatattcgatgcaaacaaagcagtttgtcatgtcttttgttttgagtctctttagtatatgctgcttttgaagatcaagaacatttactggaaattgctccttcaccaggaatttgctcacatctcttcaggtccacttataagatcttgaaatcagtcctcctgagatcagtacaaacgaggatcatctctcctgctttagaagcaaaagtggcaagcagggtagacagaggcagaaggaaggatggtactgataaatcagccaaattagatggaagaaagcgaattacagctgtacttgcaaagaactcttggctcattttttcccttaaatattaaatcactagttttcaaaacttcaaatgtgaacatgactcacctgaggacaatgttaaaatgttgacctccaagaccctgcccccaagtttggatcccaggcagaaccctggaatctgtattttaaacattaaccccaagtcgagtgtaatgcaagtaaccatcagagacactctgagaaacagcacattggcaatgatgaatttatatgtctaaaaatgaatagagtagatgttacatgaataggaagtggtggtattcaagatgaccatagtatctagctcttcagccctggctccccactgctcttctcccatctcagcacttctccatctatttcgtccaaataaaaaaattcacacagagtttcaggacttaaccccccactcattaaccatctgttttgctttacatatttttctgaggtaataaaatttctctttttctaaacacagcctgtttttcaatctccgggtagttgatcaattgtatgggaaaatgaatggctgaagggtagaaacaggtgggaaagatgaacaaaaacacgaatcctcacattactaatacgcaaatcactgagcagcaagctgagcaaataccctcaattcccatcagcaactttagagaaaggcaaattccgtttgcctcattgatcatttaggtagaccctgaacactgctttcataaaacaaaaacaaaatacccatccccagtttaaaaaattattcatagatcatccaggccatctaggaggatatgatttaatcctggctacttggtaaattatttgcccaagttaactcagctagttagtggtagatggctctgaagccagttgtttttttttgttttgttttttgcagacctcaagagtcatgatgaactagcagatcataaagtttatgccctgggtcttgaccatttttagaaaaataaaacattaaatgaaaatatcagagggcattgcagatagtagatctaagtattttttcatgaaacttgttgtacatgtgtgtgtcatacacagactatatatatgcagtacctgtaaactgtattgccacataatgtctatattttcctagaggtcacagtcaccaaagttgggaagtcacccaacttcgggaactttgggaagtcacccaaacttacagtcaccaaaattgctctattctactatgtgacctcaaaagtgatttgaaagaaggaacatctgagctgggcccaaaccctattgcaattttattggggccaaagagaactccatgctcctgccaaatcaaggcagtgtcagcctcaataatttcccagataaaaataaaaatctgtgatacaatcagaatgtgaaaattcttattttggaagcaaatgtcataaccaatgcaagggctatctcaatattcattcattatgcagtattttgaactgcagttgaaatgaataagaaggaaaggcaaacaacgaagagtccaatttctcaatttagaaaaagagaaaaaaaagaaaagggagcacacaggcacggtggctcaagcctgtaatatcagcactttggcggatcacttgaggtcaaggagttcgagaaaagagagcacctagaagttcagcgcgggataatacttaagtaaattatgacaccatcgtctgtcatcttgggcccattcactaacccaaagctttcaaaagggctttcttaaccctcacctagaataggcttccgcagccttaatccttagggtggcagaatatcagggaccctgagcattcttaaaagatgtagctcgggatgggaagttcttttaatgacaaagcaaatgaagtttcattatgtcgaggaactttgaggaagtcacagaatccacgatttaaaaatatatttcctattatacacccatacacacacacacacacctactttctagaataaaaaccaaagccatatgggtctgctgctgactttttatatgttgtagagttatatcaagttatgtcaagatgttcagtcaccttgaagaggcttttatcagaaagggggacgcctttctgataaaggttaaggggtaaccttaagctcttacccctctgaaggtaaaatcaaggtgcgttcagatgttggcttgttgtaaatttctttttttattaataacatactaaatgtggatttgctttaatcttcgaaactcttcccggtgaaaatctcatttacaagaaaactggactgacatgtttcactttctgtttcatttctatacacagctttattcctaggacaccaacactagatacctaaactgaaagcttccgccgatttcaccgaaggtcaggaaagtccaacgcccggcaaactggatttgctgccttgggcagaggtgggcgggaccccgcctccgggcctggcgcaacgctgagcagctggcgcgtcccgcgcggccccagttctgcgcagcttcccgaggctccgcaccagccgcgcttctgtccgcctgcaggtagggagcgttgttcctccgcgggtgcccacggcccagtatctctggctagctcgctgggcactttaggacggagggtctctacaccctttctttgggatggagagaggagaagggaaagggaacgcgatggtctagggggcagtagagccaattacctgttggggttaataagaacaggcaatgcatctggccttcctccaggcgcgattcagttttgctctaaaaataatttatacctctaaaaataaataagataggtagtataggataggtagtcattcttatgcgactgtgtgttcagaatatagctctgatgctaggctggaggtctggacacgggtccaagtccaccgccagctgcttgctagtaacatgacttgtgtaagttatcccagctgcagcatctaagtaagtctcttcctgcgctaagcaggtccaggatccctgaacggaatttatttgctctgtccattctgagaacccaaaggagtcctaaaagaggaatggaggagcctaagaataaaaatagtataataaaacatttcttagacacattgaccttggcctatgtcaaagttcagtctgggtttgtcttataacacaaggagtaaaagtaccattgttctacctctttttttaatacttgaaaaaaatttactgtggatgcttttctatgaattaaataaccttctaaaaaatgttttcattgctgcattcgattagattgggtaactaaatgaaattaattcctcactgttgggtataaaggttatttacagtggttctgtcttagccattcactgaactcattgcatatatatctctggaatattgctgattgtttccttcaagtaaacttagaagtgtaactacttagtcaaagagcctgaatattttaaaggccttttgaagaaaactgaaaatgctttccagaaaggatgtatcagttgacaatgacagtcgtcaacagtatttaaggagaactatgatactctgaagaaaaacttagcctttctcagtaaaagtaggtaggcagaggccacatgacagcagttagagtgtggtcttcaaggaagtcacagaaatactgtggggaattgaaaccccatgtggaaaatgtacaagagtgtctcagtgtgactgagaaggaggttgggcatggggtttcatggagtttaataaagtttggtcacttagtagaggtttaataaatcaactgtcttaatctttgatcctacttaagaattttttttttgtttttgtagagatggggctcttgttatgttgcccaggctgttctcgaactcctagcctcaggcgatcctccctcctcaggctccagaagtcctgggattactggcgggagccaccatgcaggcctcttgctcctacttttgagaaaggaagtttaaccggttttttttgtctttttttttttttttttgagacagagtctcactctgttgcccatgctggagtgcagtggtgcaatctcagctcactgcctcccgggttcaagtgattctcctgcctcagcctcccgagtagctgggactacaggcacctgccaccacgcccagctaatttttgtatttttagtagaaatggggtttcaccatattggccaggctgatctcgaactcctgacctcaggtgatccgcctgcctcggcctcccaaagtgctgggattacaggcatgagccactgctcctggctgcttaactttttctctatctcatcctcctacccatcctacccttggaagatagagaagtagtattagttccatagtgttatactgggcttcccccagggacaaacccacttccccaacctgaatgagccatcacttcttccccagtttacatttcattgctctttaaatgtctccattcggatatgggaattcacatatggtcataattcttacctgaagaagatgtcagtcttcttctcttagaccaactgccctgatatgaggtttagaggttaaagaacatgtgtgtatttacatgatctttgtattctgccttttcgtccctcactaatgacagctgcaccccaaggaaatggagctgtggaagagagggtttgataagaaattaagtaaatattggatctaatccatcaccctccaggaagcctttattactcctaaaaatttcaaccaaattcattaaaggacaagaactccaccagagtaggccataaacattggcaaaattagttgtaatccatgactagatttaatgtccctttgttttattcccatatggttataatgctttgcttggcattaggggtattttaagttttcttctgcctagtaagtgaatttgtgtttataatacaataatcataaaatatcacattaatattttataactgtacagttataaaatattttataagtaatatttatattttataagtaatattttataactgtacagttaactctggcccaaggaaaagatagtctgatagatgctgcagccccattttagcaaatgtgacctcacaggcctgaatgccatcgctattccacatctacaggatagacggaaaggaaagaaataaaaaaataggtacctaacactggcaagaggatgatgactcatgttatttcacttaacctttttatcttttaacatgaaggactcatacaggttgataagaaaccagtgacataaacagaccaaaaaatgatcagatctttcaaattagcaaaaaaataatattttttaaacaatgggtgaaaatacagtgtaacagtaccaattatcaacatgtgttgagaaccagaaaaatgttctttttctttgatcagcaacactatttgggaaaatctatcctcagggcctagcctggggccctggcacacagtaggcactcaacgaatatttgctgaacacacaaatacttatgatattttaaaaaattggcaacaatctgatacctaacaatagagggattaaatattatggaactgttaaataagatgcttatgaataccatgcagtaagatgggcaatatttatgccataagctttaatgaaacaaatgggtattaaatgtatgataaggttataaattactttttaaaagattacagggaaaaaaattgaaagatatacactgaaatgttttttgctcacagtggtgacaaggtttctcagcactggcactgttgacgttttaggctgtatgtctttgctgtgggaggctggcctgtgcactgcagggtgtttggcagcactcttggcctctgcccctagatagcaatagcagtcctccctcaaccagcccaattttgacaaccaaaaatgtttccaggcatcaccagatgctccctgggtgagagtgatgaaatagtaggggattttccccttcttttcttattttctgtaattccattatattactttaataataaagaaaaaaacataaaaaataaacgaatgttattattctacgtcagtttggatgtttggactccattttggggttctttccattatatcacttggtctgctaaacattctacggtttggtaaggtgaagtgattcatgaaattttggttttatttttttcctgatactaaaaataaaacattctttcacttggaaatttggacacagaacaccaaaaaaaatccataatctcatctctctttttctgtcttttccttccttttttccctttaaaaacaataaagagtgaaacctacctgttctccctctaatttaattcctaaatataatcactgtcaatatcttggacatttcctgtgtctaaacacacacacacacttttttttttcagcaaaagtggatttctgctacatgtagtgttctgcaacttactttctatgtgtttacaaaatcagtacatgtacatatgctgaattcagtccttaatggtattatattttgtgaatataccaaaatttgtttaaccacttagacaatctaggatattctcagtttgctgttatgagcaatgctcttcctttacatatacagacatatatatatatatgtgtgtgtgtgtgtttttgttttagtaggatagatttctaggagagggtgaaaggtcttatgacatccgcatttacgattgtaataggaagtatcaaagtgccccctaaagaaaaaaatcctcccattagtgggtaagaaagcctatttgttcatatcttcacaaacactaaatattagaaatatttacaattgtggtcaagctcataagtgaaaatggtatttcatatcttatattttttattgtgagattgaacatctttcatatgtttacatgtcacctgtatttcttattctctgaactatatgttatgacctttcactttttttcctcatgggttatgtgtagtttgtatagttgtcttattgattgttaggagctatttatatattaggaacattaatctcctgtcttatatatacgtggcatcgattagttgatcatttgtgagttcatgtctgtatacaaagattggagaggcactaagagggaaaacttacctctttcttatcaaagtttgtaaatatatgtataacagaagagggagaaaatattaataaatgcacagattggctgaaatagagtataaatcttttactcccctacttcaacataaactgcaaaaggagagtgacttttctttcactctgacttccgtattcctcatgcttaaaatagtgcctagcacagaagaggtgctcaatcagtgtttgctaaacgaaataattagtcacatttcaagcaggatgactaaatgaagaatagaatctaggcagatactctggaagagtggctgtgagtcattcatatcttagtatgaattagtcaaatccaactctctccccttcccactccccactgttagtagaagaatctgtttattgagagaatagatttataatttagaataagtgagaggggcagaagaggagattttgaaggatggcacctgaaggaggactagcatggctgagacagtgaagtggaagccttgaatagctaaagggtaagatgaaagtatttagctgtagggggaaaaagcattgacaggttggaaaagtaaaagtcagattctccttgctctgaaattttgtacagggcaggttctactaggtatgttacaatgcagaaaaaacatgaaataattgagaggaatttggtgcaatattatcttcttggcttcttttgagtgggcagatttttttcacggcctgtaactataataaatttgaaacttctcatcttttagtaacttttttcacttaagtttatgtggctgtgggcaatggaatgaagatattgaacttccaattccctgttgggtttccacaattacaagtcaatcatgactggttattagaagactatttcagttagaaccaccaagtcccatattgtcatattgtatgtttaattattaagtgaagcagtcttcttttcgtgttttccataattagggcattccagaaagatgaggatatttgctgtctttatattcatgacctactggcatttgctgaacggtaagacaccaaatccttccattaggttctatattttaaatattttaaccatgagtttaaaactaaaatgatcatttaaaatgcatgcaattttcttatagagagaacattctattctttcttctactttacacaatggcaaagtcttctttctactttacgcaatgataaagttacctgtgtcattttgtaaaaatatagagaatatagacaaattgaaagacacaaaataatctattacccatttcccagggttaactactgaaaatatctggggaaatggcctgtatgtatacatttatttgtttgctttcaacaaggccaagatcctttgatctttcagtcttggttgctctgtgacatgcctttcctgatgaggatactttaaggaagaattgtaagatacatggaaaatgtcaggctaacacagtactggcatcaccctgtgctctttcctgaactccataccaatgtacttcttgccagaaaactgatcaaaagtttagggaagtaaaaagagatgactgttagaatctaccattccctctatgtaggaagcaaataggtgtcctgtcaaaggacattctggggatgtctacatgaaaccaagtctccctggttgtaaggactccatctccatataatatttatacagtaatatatgtttataaattgtgggggcaacttgtttagctaattttattattctgctattgggacactgtgtctcagcatgagatatagtgtcccaaaacatatttcaagcccattggataaaatatgtgtttagcaagttcttaaatataatgataacataaccgaccagataaagtgatttataaacgctgtgccaattttgtaaatgtttcgaggaattttcccttttctgaagattgtccttctttctttttagcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatggtaagaattattatagatgagaggcctgatctttattgaaaacatattccaagtgttgaagacttttcattcttgtaagtccatacttattttcaaacagaacagcatagtctgttcattcattcattcaattcatgaattcattcacataattatccaatttcttgagcacctatttgatagtcactggaaatccagagacaaacaacacagagccatgttctacagtatgtacagttttccaaaaagaatttctagtctttacttttttattacaaatggaatacgtatacttgcaaataattcagatactgtggaagagatcaaatgaattgcaaaagtgtccctcctcccttcaccactatctcccatggcatgcagagagagtaaccattatttgtgtgtccctccagaaatttttttattcaactactatttttttattttattaggtccgtcagttttccttttttgagcctctctatatcaaatgcaaataaatatattcagaacaaaccccactgtaaggttcacattaaaaaagacttgaagtcaccctatgaagacaaaaaataatcacattaagtgtgaaagaacctattcttccagtacaggataagccatacttactgggcatatattcatcttgaaaatctatactgatgttgtcttggggaattgaaaaggaactaggagtgttagttcctcggtattgacccacagttatgttatcaggtcacttgagttcaaagttttgtgttggcactagctaagtaaaggaaaacacctctgctttcattgttgagtttcacagaattgagagctgaaaggatcccaggcaggagcagctaatccaaactcccacaaagaacaaaaatcccccagaggatcttctgttcttatatttcctgcaatggcgtccctgtcatatcccacaatggcctccctgccatttggatatcccttccatatcctgttgaaattactccctaatagtaagctgaaatctgcccctctagttgtagtcttgggattatttcatttacatgatgaccttttaatatttgactagaattaaatcatctccccttggtctttccattcctgggctaactaccatcaatctgagggctaacaatacaagtagaaaaagtatacatttgtcactgatcactgatcaattattaatcaatgatcactgataactataaactcaaaaacaaaatcatgtggggattaagagaaatgtatcagttttatgttgtatttctggtccctgatactggctcaggtaatgccactattgtcaagaagataccacttgtaaagtagatttaattttcattatattttaccatatgcttctccattcatgacatctcttgagatgttgtggtttatactttcagtttttctccagtccatccgcaaatatcaggcatctactgtgttccaagatattaaagaaatcatcatgacttagcctcatcaacagcattgctagatctgggatggaaaggaagagtataatcctggcagtcaggaagaaggcagcataaagtataagtttctgcttccaaaaaaggtctctcatcagcctgtagggagtgtgtagggaagggacagctgtccttgtagtagggaagggttttattcaggtcgtctgggctccataatatcccttgtgtatctgcagtctcctttgccatggatcaacacaataggaaatcttccggcactgatggtttttccaagggggagttcttcctggagcaaagcaaatgaccaaccaggtttgaggacctgatttgtttgacaattccattttgtattgtaaattacttaattggcattctactcccaatccatcttgtcatttgcatacagtggttttgggattgagttcagctataccaaaagtctgaaccttctgcacttagaacaaggcaaccaccaagcttcacttgcactgaggccgtgtctccaatggaaatgaggcagctggcttgcaggagcttcccaactcagggaagtagaactcctgagtcacctccatatgcaaatgatttcacagtaatgctgttgaacttcacttcccatcacagcaaatgtgtggtaacatagcttccccacaggagtttactcaccatggtattttaaaggtgaaacatttcaaaactgaaatttgaaagaatttagttttggattcactcaattatcactatcacttcgggtgttattgcacctttcttgtttgtgagtttaaatgccagactctcaggccactaactttcaattaaaagtgtttttctttaatcgctgaacctaacagcagggaaaacgaaatgttcattcagactttcagaaccttcaatgagattaggcagctgaaagatcaaagtgttgcatagttgtcccgataaagctatttggatcatatggaccaaatcgactgctgtcattccccaccaaccccatctctccccaaaattcccagccctgtttaagtgttctctgtagcatttatctctatctagtatattgtgtagcatatcatatcatacttttctgttttgtttattgtctctctcctcctagaatataaactccacaagcacaaagatttgggcctgttttataatattgttgcatccccagggcctgatatacagcagagtggtggtacgaaaagagcacacaaaaaaatatttgttgagtcaatgaatgaatgatttcctcaaataggattagcctaaaattttggaaacatgaacagatttggatatgtgaaaatttatttccagactgttcatcaggaactgttagcagcttctaaagggtacactggagcagcagtagtaaaaggaggaagaggagcagctctgctactgctactatcgagtactactacaattagcacttgcttattctgtgtgttaggccctgtactgaacactctgtctaaattagttcatttcctcctggaaatgactctagggggtaagtgcttcatcatgtaagatgagtatttttcacattttgttgtgtctgaaatctgagtgtgtctttcaatgatggaatctttgattccatgataagtggtattattcccattttaaggatgaggaaactgaggtccaaagaaattaagtaatttgcccaaattcacccagcctagaaaatgataaagctagttctaaacccaagcagattagctctgaagtctgggcccttaataaccactttttattgcctatatttgtacctctggtgtacgtatcaagttatatgttgacttcaaaactatcatgaccttttcttggttttgattgtccaacattagtatagtgttctgggtctgcaaaaattttgattactcatctcatctgtaaaacattttgaactcgtgtgtttgtgcatgcacatttgtgtgtaattataaaaattttactttctgttaatatataagttgtatcataagaaactgccgtttttgaagagcaaaaaaaggttgaatgttaccagttacatctggttcaacctaatagacatttgtacaaaaacagacattttaagaggttgaaataaaaatttaataaacaatattttcagtttttactaattgtgatgcttcactatcattagctaatatgtcaaggcataatataccttagggtgaactttatcattaacaaaggtggatggtgtcaataatcttgaggtttgtgtttttttatataacactgcgaggtctaattaagtacttactgtttaccacctcatacagtggccgataaaaagtgtcacttctgctgtttcctctgggttgtgcttgaattattagtattatcttcagtcctcagtttctttgtgggaaactttttaattagttgtttaattttgtaagatggttagtttagtcaaaattagataagagaatttgaaaatccgtagctaccccaaagcaacctacacataagaactattatttttgtgttttgaaatcataattttattgatttccagtgtttccactggtagtggtttcattgatataggagtatcaaaacatcactcattatttatttcagtttcatttgatcctagccgttttgtattaactctctgtgaagaaattacctcacaaatctattgctgtccttggtaaaggaatggagaattaaggctctagatcattagtggttacactatagtattagaagtaaaaaaaagattataccaacaaaataagaacatgttaatgtacttgtaatgaataaacatgaataaagctcttatgctatataggtgcactaaacaatctactagaattgtcagcaaactacgtatcttaatcctgaaagggtcccaaaccaatgatctaaaattgaatcaaactttcttccttgagcataattacttaaatgatttattaaaatagccagcatttaaaagcttaaaatgtaaatatcataatgtggtatcctagatagcatcccagaacagaaaaaggatattagggaaaaactggaggaatggaataaattatgcagtttagttattaataatgtactaacgtccttagttatgacgattgtaccatggtaatgtaagatactaacaatagaggaaaccgggtaaggagtatacagtaactctatactatctttgcaacttttttgtaaatttaaaacttctaaaataaagaacaaatttaaacattaaaaagtatcaccaggaacatatatcactgtttacagatgaaatactatgtattttcatatctaatttctgatcattgacttcaaatcagaaaagtgaatgacacctcaaaatcaggttttctgtttactgaagtctaagaaaagaaagcataccagctggagagattcatgtttataaagacagatttataacaacaaaaataaaatatccaagaataaatttaagaagaagcactttactgagaaacatatgaaaacctgaacaaatggagagggatattttgtatttgaatagaaagacttctggtttaaagataattctctttaaattattttttgtagaaatttaaggggtacaagagcagtgttgtcacatggatatattacatagtggtgaagtctggggttttagtgtaaattaatctttacattttgtttgagcccaataaatgtaccaacatgatttttatagaaagatagtcattcctattaatccaaacttgtcccaactttgaattgaattgaggcagagctagcaggtgttccccacggctgaggcatctgaacattaagcatatccctctgagaaccagcctgcattgatactctttctaatgtggacagcatcaagctatgtacgtagttctgtgctcagcaaaagccctgacttctttttgtttatgtcctagccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataacactatgtttacaaaatatatcctaattcctcacctccattcatccaaaccatattgttacttaataaacattcagcagatatttatggaatataccttttgttccatgcattgtagtactcattggatacacatagaataataagactcagttcacactcttcaggaaacagataaaaaactaagaaacaaacaaaaaacaggcaatccaacaccatgtgggaaatgctttcatagccgggaaacctggggaatacctgagaggaatactcaattcaggccttgtttcaggaatccaaatcctggcacatcagagctgcttccctctttccagggtggcaggaaataaatggaacatatttttctatcttatgccaaacatgagggaccctttctccccggtgcctctcccaaggtagtctacaatatttcaactctagcagtctgcttagtgcatagaacatgaggctgtgtgtccctgggcaaattactagacttctgtgtgcttcactttccctgtaggattataatctactgagcaagcttattgtaagggtcagattagcaacagtgtatgaaaatgatttgagaccattgcctgcacaaattcaactatttttttttatctcactactctacagaagtaggtagggtgggagacagagtctgatgagaggctcagaatgtgaaagaaagtgaggcgagtgagcatgatatttaatataaacacaaagatattctgagaagagctgctcactgccccctcccccaatacatgttgataggaaaatgccacgtacttcagcaaaaacaactgaaaaattagatagaaaagtcaatcaataggaaaagataatccaggacggtgttgtgaacagaaagagggggaaaaaactttagaaaatgatggggatgctcttactggggtacgagtcctcaggtattgaactggctttcagtaaaagctagattagtgggttcctgccatttacaagctgttttatgacaacttacttgttgggtggcctacagtaactcacctaactgcactgagtctgtttcctcatctgtaaattggggatttttttttaaatacctggcatgcctaactcataaagttgttctgaaactgaaataaaacatacgtgaacaggcattgtaaactgtaagttacggaaaaagctggctgttgttgtgtctttaaagtttcacctgggtagtcaaagatggatcatgggtctcagtggagagctgagccaggcaggagctgactaagggtgagaggtgggagttagcagcctctgaacatctgtgtaccatgggaccccctttcctcctgcatggtaccccagacaaggagcctagtaagagatactaatggcttgttgtccagagatgttcaaactgcagagaaagataagacaacaagcattggcctccaatcatgatgacagataggaggaggtgggagctccttagcagtgctggttggccttccatgttctactgtgggccatctctgccatgtactgtaggctactagcttctatattaaagaatgcaagaggggccaggagcggaggctcatgcctgtaatctcagcactttgggaggccaaggtgggcagatcacttgaggtcaggagtttgtgaccagcctggccaacatggtgaaactctgcctttactaaaaatataaaaattagctgggtgtggtggtgtgcacctgtaatcccagctactcgggagactgaggcacaagaattgcttgaacctgggaggcggaagttgcagtgagcccagattgcgccactgcactccaccctgggcaacagagaaagactctgcctcaaaaaaaaaaaaaaaaagcaagaggaagtgaaataatcaaggccgccatttaatagtgagcagccactccatgtggtactgtgcaagcacattataaatattagcctcacaagaaatgtattagcatttgtattttgtacactggttaagtatcttgcccaagacctcaaaactggttaagggcagcagaatttagccccagcaccaccttttcaaagcctgggcttctcacacttctccatgctgttcccattttaacacaggtatctcgccattccagccactcaaactttggcatttaagaaaattatcctaaagctaaactaaacttcaaggatgaccattctcctgaccccttcccatcaaaattttatctttagtcagtttgttttcgttttgttttgtttttcagaactacctctggcacatcctccaaatgaaaggactcacttggtaattctgggagccatcttattatgccttggtgtagcactgacattcatcttccgtttaagaaaaggtagtatttccttaattgcagtggtctccactgggggtgaggaaggggtgagaattggatcatggctgcaaggaaacccgacttaacctctgcaaggtggtgcaaaggcattccactgttcaacagcaattatattgaagctgagtgggatcactgggtgaagatgaagcgtaaggggtgaggggcaggagaatgggtatggatggaggtagaagatgcagtgtcatacagtttttttctatcatgaaaataaccacagacttacagaagagaaagagctaaaatgcccgtcattttcagttgcattttagtcttgcattagttgcaaccagctggtttctgggtaccctaagtaataaaaatagttcctctgtagaactgtagtatgtttaccatagagtattttgcaaaatttttggtagaggatgttacataatttgcatgtgttcatttctccatttacctgtgggaacaattaaaatccaggaaaatgagtatattcaaataatttcctcccatttaagatgagtcagagtaaataattcctccaatacttagagaagtataccaagagatccagtgatggtatagagttgtctgatgttaaatagggaagtagaatatggaaggggattccaatagtcgttgaaaaattccccataaccccttacatgggggaaagtagtgttaactgagagagtagagataagctgtttccaaaaattatattcttaacaggactgagatagccagaatataaggatcaagtttcaatgacagtaagatcctgagatggagttgatttgcacaaagaaataattgttgccagcatgcattttgaatatttctctggaaaaaaagattagttggcagtagaaatggatagaaatcaatagatattaaaatacctcagaatttggttcatctctgggaaaagatgaaaaataaaagtgtatactcctcaagaacatctaggatcaaaagcatgtgccctacactattgaattaattaacctcataagttgggacctgtggaataaggatgtccaccagacttcctagggattacaaatgtttcacagaacttgaaatttaaacttgggtcactgtatgggatgtagagctgtgctatatggaaataaaaatgatttctttttctcaagggagaatgatggatgtgaaaaaatgtggcatccaagatacaaactcaaagaagcaaagtggtaagaatatcagaaggaattgggaagtaaaagtcaaaggaaacaaaaagctaaagcaataacaaagagaaatccatcagtcataatctcctctccttttaaagaatgctggttcccctttgcctcacagctaacacaagaactcctccaccgtctgaggaggtttaggagcagggaaggggaaggagtcagcttcatttgctaatcttctgttgccctgcaccctagcagctccttgcagcaggggacaaggatgacttaggtggatggataattaattgattctaaaatattgtgtgtcagtattgtaatactatgttaattgcaccatgcacggtatctcatttaatcccccaccccttgccattaccaaagagagagagagagagagagagagaaatactagaatttatcctcattttacagtagagaaaacagagggtcaagaagataatgtaaagtgcccaagaacacacagctgatcacaaaaatcaagcttgggggccattagcctaaccacagacccttactcttaacccatctgcttcaatccattttgctacaaatgtttacatttataagcagggcagaaaaacctcatccaggttattgaactaagaagaaagttatattaaggtttctaatttttttaatgtagttagaaaccaaacttaacaatgagcccaagtttaaagcagtctaattaacctggacaagctcaggcaagtttcattctgtggcccatagcatcatctgtgttgtaaagctaagtagcaaatgttgtttgggtcatgctgggggacaagccatcccaatttgctcaggactgaggggttttccaggatatcatgtaaggataattgggtacaaatataacctgctgctttctctcatttcaaatttatcatttatcatatcagcaactatgagttatgttttttattagatttcttgttactttttccccagaccacttcccatgaaattaatatactattatcactctccagatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttacttggaatatttgcagccttgttcctagttcttttttcattgcttatacagcatgtttttatacagcatgcttttgaatcctgcacaatgaaaaaagtatcaaaagattttattttaaaaaagaatgtattgaaaattttcattctctctcattcggcagcttcctttaggaccataccaagaagagtcaaaatcctgtcatctacattgaagatctaagggagagagggtgggagagaaggagtggaacccaatagaactatagggaaacaaagggacttacctctaagaaagtacttttcccagatctgtaaacgggtatgcaatccagtgtgtaggaagatagatgtattgcagtcttcaagttttgaaaataaagcgaggaaactagaactaagccaagttgaggattaagggctagcaatggttaggaccatagatctgtggttttaaacaagcatgacaaaacttatcattcaaggaagtatagaccttttaatgtgtcatccggtgagattttagtcatgaaattccattttaaaagactatcacttatttgaatcagatactcttaaaggattataatggctatttctaagaatatgtgtttgactctagatgcttttgtttgtttgtttttgagacagagttttgctcttgtcacccaggctggagtgcaatggcactatctcagctcactgcaacctccgcctccaggttcaactgattctcctgcctcagcccctcgagtagctgggattacaggcacctgccaccactcctggctaatttttgtacttttagtagagacagggtttcacaatgttggccaggctgatctcgaactcctgacctcaggtgatccacccatctcagcctcccaaagggctgggattacaggtgtgagccacggtgcctggcctctggatgttttcagtagtaatgatgatgcactatttatgctagcccttgggcagtcaaagcaataaaaatgtgtgctagacatcaagatatctggccttcctaatcaggaaaggatatggagaactagtcttcctggcttgccttggtggtatggaaattttgtgacacttgaggggtctgtattatagaaaagttaatatgaaaatggttcataagctttcattaaacttttgttctagtcccttttacaagtgtgtgcctgtctgccacaagtaggcctaatatattttttaagttttatttttaattgacatataattgacttttttttattattatactttaagttctagggtacatgtgcacaaaatgtaggtttgttacatatgtatacatgtgccatgttggtgtgctgcacccattaactcgtcatttacattaggtacatctcctaatactatccctccctcctccccgtaccccacgacaggccccggtgtgtgatgttccccatcctgtgtccaagtgttctcattgttcatttcccacctatgagtgagaacatgcggtgtttggttttttgtccttgcaatagtttgctgagaatggtttccagcttcatccacgtccctacaaaggacatgaactcatccttttctatggctgcatagtattccatggggtatatgtgccacattttcttactccagtctatcattggtggacatttgggttggttccaagtctttgctattgtgaatagtgctgcaataaacatacatgtgcatgtgtctttatagcagcatgatttataatcctttgggtatatacccagtaatgggatggctgggtcaaatggtatttctagttctagatccctgaggaattgccacactgtcttccacaatggttgaactagtttacactcccaccaacagtgtaaaagcgttcctatttctccacatcctctccagcacctgttgtttcctgactttttaatgatcgccattctaactggtgtgagatggtatctcattgtggttttgatttgtatttctctgatggccagtgatggtgagctttttttcatgtgtctgttggctgcataaatgtcttcttttgagaagtgtctgttcatatccttcacccactttttgatggggttgtttgattttttcttgtagatttgtttaagttctttgtagattctggatattagccctttgtcagatgggtagattgtaaaaatgttctcccattctgtaggttgcctgttcactctgatggtagtttcttttgctgtgcagaagctctttagtttaattagatcccatttgtcaattttggcttttgttgccattgcttccagtgttttagtcatgaagtccttgcccatgcctatgtcctgaatggtattgcttaggttttcttctagggtatttatggttttaggtctaacatttaagtctttaatccatcttgaattaatttttgtataaggtgtaaggaagggatccagtttcagctttctacatatggctagccagttttcccagcaccatttattaaatagggaatcctttccccatttcttgtttttgtcaggtttgtcaaagatcagatggttgtggatgtgtggtattatttctgagggctctgttctgttccattggtctagatctctgtttcagtaccagtaccatgctgttgtggttactgtagccctatagtatagtttgaagtcaggtagcgtgatgcctccagcttttttcttttggcttaggattgtcttggcaatgcaggctcttttttgattccatatgaactttaaagtagttttttccaattctgtgaagaaagtcattggtaacttgatggggatggcattgaatctataaattaccttgggcagtagggccatttcacgatattgattcttcctatccatgagcatggaatgttcttccttcggtttgtgtcctcttttatttcattgagcagtggtttgtagttctccttgaagaggtccttcacatcccttataagatggattcctaggtattttattctctttgaagcaattgt

SEQ ID NO: 2 - DNA, 17 bp, Artificial Sequence
  antisense sequence
ttaccactcaggacttg

SEQ ID NO: 3 - DNA, 17 bp, Artificial Sequence
  antisense sequence
atgctggattacgtctc

SEQ ID NO: 4 - DNA, 17 bp, Artificial Sequence
  antisense sequence
aatgctggattacgtct

SEQ ID NO: 5 - DNA, 17 bp, Artificial Sequence
  antisense sequence
atgatttgcttggaggc

SEQ ID NO: 6 - DNA, 17 bp, Artificial Sequence
  antisense sequence
ggcgacaaaattgtaac

SEQ ID NO: 7 - DNA, 17 bp, Artificial Sequence
  antisense sequence
gtttagtttggcgacaa

SEQ ID NO: 8 - DNA, 17 bp, Artificial Sequence
  antisense sequence
cacaacgaatgaggctt

SEQ ID NO: 9 - DNA, 17 bp, Artificial Sequence
  antisense sequence
tagactatgtgccttgc

SEQ ID NO: 10 - DNA, 17 bp, Artificial Sequence
  antisense sequence
tgagtagactatgtgcc

SEQ ID NO: 11 - DNA, 16 bp, Artificial Sequence
  antisense sequence
tggattacgtctcctc

SEQ ID NO: 12 - DNA, 16 bp, Artificial Sequence
  antisense sequence
tgctggattacgtctc

SEQ ID NO: 13 - DNA, 17 bp, Artificial Sequence
  antisense sequence
cgagctagccagagata

SEQ ID NO: 14 - DNA, 15 bp, Artificial Sequence
  antisense sequence
ctagaccatcgcgtt

SEQ ID NO: 15 - DNA, 17 bp, Artificial Sequence
  antisense sequence
aatcgcgcctggaggaa

SEQ ID NO: 16 - DNA, 16 bp, Artificial Sequence
  antisense sequence
actgaatcgcgcctgg

SEQ ID NO: 17 - DNA, 17 bp, Artificial Sequence
  antisense sequence
tacctatcctatactac

SEQ ID NO: 18 - DNA, 17 bp, Artificial Sequence
  antisense sequence
tggacctgcttagcgca

SEQ ID NO: 19 - DNA, 17 bp, Artificial Sequence
  antisense sequence
accggttaaacttcctt

SEQ ID NO: 20 - DNA, 17 bp, Artificial Sequence
  antisense sequence
tatggcctactctggtg

SEQ ID NO: 21 - DNA, 17 bp, Artificial Sequence
  antisense sequence
ggaatagcgatggcatt

SEQ ID NO: 22 - DNA, 17 bp, Artificial Sequence
  antisense sequence
tccgtctatcctgtaga

SEQ ID NO: 23 - DNA, 17 bp, Artificial Sequence
  antisense sequence
tccaaactgacgtagaa

SEQ ID NO: 24 - DNA, 17 bp, Artificial Sequence
  antisense sequence
caccttaccaaaccgta

SEQ ID NO: 25 - DNA, 17 bp, Artificial Sequence
  antisense sequence
atcgtaaatgcggatgt

SEQ ID NO: 26 - DNA, 17 bp, Artificial Sequence
  antisense sequence
tcctattacaatcgtaa

SEQ ID NO: 27 - DNA, 17 bp, Artificial Sequence
  antisense sequence
gagcttgaccacaattg

SEQ ID NO: 28 - DNA, 17 bp, Artificial Sequence
  antisense sequence
atcgatgccacgtatat

SEQ ID NO: 29 - DNA, 16 bp, Artificial Sequence
  antisense sequence
actaatcgatgccacg

SEQ ID NO: 30 - DNA, 17 bp, Artificial Sequence
  antisense sequence
tcaactaatcgatgcca

SEQ ID NO: 31 - DNA, 17 bp, Artificial Sequence
  antisense sequence
tagttacaggccgtgaa

SEQ ID NO: 32 - DNA, 17 bp, Artificial Sequence
  antisense sequence
tatagttacaggccgtg

SEQ ID NO: 33 - DNA, 17 bp, Artificial Sequence
  antisense sequence
tcattgcgtaaagtaga

SEQ ID NO: 34 - DNA, 17 bp, Artificial Sequence
  antisense sequence
tatctggtcggttatgt

SEQ ID NO: 35 - DNA, 17 bp, Artificial Sequence
  antisense sequence
atcactttatctggtcg

SEQ ID NO: 36 - DNA, 17 bp, Artificial Sequence
  antisense sequence
cacagcgtttataaatc

SEQ ID NO: 37 - DNA, 17 bp, Artificial Sequence
  antisense sequence
attggcacagcgtttat

SEQ ID NO: 38 - DNA, 17 bp, Artificial Sequence
  antisense sequence
actgacggacctaataa

SEQ ID NO: 39 - DNA, 17 bp, Artificial Sequence
  antisense oligonucleotide
ccgaggaactaacactc

SEQ ID NO: 40 - DNA, 17 bp, Artificial Sequence
  antisense oligonucleotide
taccgaggaactaacac

SEQ ID NO: 41 - DNA, 17 bp, Artificial Sequence
  antisense oligonucleotide
ggtcaataccgaggaac

SEQ ID NO: 42 - DNA, 17 bp, Artificial Sequence
  antisense oligonucleotide
acgccattgcaggaaat

SEQ ID NO: 43 - DNA, 17 bp, Artificial Sequence
  antisense oligonucleotide
gattgatggtagttagc

SEQ ID NO: 44 - DNA, 17 bp, Artificial Sequence
  antisense oligonucleotide
gcctgatatttgcggat

SEQ ID NO: 45 - DNA, 17 bp, Artificial Sequence
  antisense oligonucleotide
atcagtgccggaagatt

SEQ ID NO: 46 - DNA, 17 bp, Artificial Sequence
  antisense oligonucleotide
accatcagtgccggaag

SEQ ID NO: 47 - DNA, 17 bp, Artificial Sequence
  antisense oligonucleotide
ggttgccttgttctaag

SEQ ID NO: 48 - DNA, 17 bp, Artificial Sequence
  antisense oligonucleotide
ttgcatatggaggtgac

SEQ ID NO: 49 - DNA, 17 bp, Artificial Sequence
  antisense oligonucleotide
aagctatgttaccacac

SEQ ID NO: 50 - DNA, 17 bp, Artificial Sequence
  antisense oligonucleotide
gttaggttcagcgatta

SEQ ID NO: 51 - DNA, 17 bp, Artificial Sequence
  antisense oligonucleotide
tgttaggttcagcgatt

SEQ ID NO: 52 - DNA, 17 bp, Artificial Sequence
  antisense oligonucleotide
acagcagtcgatttggt

SEQ ID NO: 53 - DNA, 17 bp, Artificial Sequence
  antisense oligonucleotide
gaatgacagcagtcgat

SEQ ID NO: 54 - DNA, 17 bp, Artificial Sequence
  antisense oligonucleotide
actcgatagtagcagta

SEQ ID NO: 55 - DNA, 16 bp, Artificial Sequence
  antisense oligonucleotide
agtactcgatagtagc

SEQ ID NO: 56 - DNA, 17 bp, Artificial Sequence
  antisense oligonucleotide
tagtagtactcgatagt

SEQ ID NO: 57 - DNA, 17 bp, Artificial Sequence
  antisense oligonucleotide
ttgtagtagtactcgat

SEQ ID NO: 58 - DNA, 17 bp, Artificial Sequence
  antisense oligonucleotide
agtgctaattgtagtag

SEQ ID NO: 59 - DNA, 17 bp, Artificial Sequence
  antisense oligonucleotide
atacgtacaccagaggt

SEQ ID NO: 60 - DNA, 17 bp, Artificial Sequence
  antisense oligonucleotide
agacctcgcagtgttat

SEQ ID NO: 61 - DNA, 17 bp, Artificial Sequence
  antisense oligonucleotide
attagacctcgcagtgt

SEQ ID NO: 62 - DNA, 17 bp, Artificial Sequence
  antisense oligonucleotide
tacttaattagacctcg

SEQ ID NO: 63 - DNA, 17 bp, Artificial Sequence
  antisense oligonucleotide
tatcggccactgtatga

SEQ ID NO: 64 - DNA, 16 bp, Artificial Sequence
  antisense oligonucleotide
gattaagatacgtagt

SEQ ID NO: 65 - DNA, 16 bp, Artificial Sequence
  antisense oligonucleotide
cataactaaggacgtt

SEQ ID NO: 66 - DNA, 17 bp, Artificial Sequence
  antisense oligonucleotide
atcgtcataactaagga

SEQ ID NO: 67 - DNA, 17 bp, Artificial Sequence
  antisense oligonucleotide
tatgttcctggtgatac

SEQ ID NO: 68 - DNA, 17 bp, Artificial Sequence
  antisense oligonucleotide
catggtgttggattgcc

SEQ ID NO: 69 - DNA, 17 bp, Artificial Sequence
  antisense oligonucleotide
ctgttgctaatctgacc

SEQ ID NO: 70 - DNA, 17 bp, Artificial Sequence
  antisense oligonucleotide
acaccgtcctggattat

SEQ ID NO: 71 - DNA, 17 bp, Artificial Sequence
  antisense oligonucleotide
tctgttcacaacaccgt

SEQ ID NO: 72 - DNA, 17 bp, Artificial Sequence
  antisense oligonucleotide
aatacctgaggactcgt

SEQ ID NO: 73 - DNA, 17 bp, Artificial Sequence
  antisense oligonucleotide
ctagtagcctacagtac

SEQ ID NO: 74 - DNA, 17 bp, Artificial Sequence
  antisense oligonucleotide
gcttgcacagtaccaca

SEQ ID NO: 75 - DNA, 17 bp, Artificial Sequence
  antisense oligonucleotide
ctggaatggcgagatac

SEQ ID NO: 76 - DNA, 17 bp, Artificial Sequence
  antisense oligonucleotide
tcagacggtggaggagt

SEQ ID NO: 77 - DNA, 18 bp, Artificial Sequence
  antisense oligonucleotide
cgtttaggctatgtactt

SEQ ID NO: 78 - DNA, 19 bp, Artificial Sequence
  R01011
gatcattcgcggacacaac